SEQUENCE LISTING (1 sequence)

SEQ ID NO: 1 - protein, 7 aa, Artificial Sequence
  Synthetic Polypeptide
YGNGVXC

(19) United States
(12) Patent Application Publication
(10) Pub. No.: US 2012/0027786 A1
(43) Pub. Date: Feb. 2, 2012

(54) GENETICALLY PROGRAMMABLE PATHOGEN SENSE AND DESTROY

(75) Inventors: Saurabh Gupta, Cambridge, MA (US); Ron Weiss, Newton, MA (US)

(73) Assignee: Massachusetts Institute of Technology, Cambridge, MA (US)

(21) Appl. No.: 13/033,233

(22) Filed: Feb. 23, 2011

Related U.S. Application Data
(60) Provisional application No. 61/307,301, filed on Feb. 23, 2010.
(60) Provisional application No. 61/382,637, filed on Sep. 14, 2010.

(51) Int. Cl. A61K 39/00; C12N 5/10; C12N 1/15; C12N 1/19; A61K 35/74; A61K 35/12; A61K 35/14; A61K 35/64; A61K 36/02; A61K 36/06; A61P 11/00; A61P 1/00; A61P 31/04; C12N 1/13; A01N 63/02; A01N 63/04; A61P 37/04; A01P 1/00; A61P 1/14; C12N 1/21

(52) U.S. Cl. 424/184.1; 435/252.3; 435/252.33; 435/325; 435/348; 435/254.11; 435/254.2; 424/93.2; 424/93.21; 435/257.2

(57) ABSTRACT

Aspects of the invention relate to compositions and methods for using recombinant cells to sense and destroy specific pathogens.

### RELATED APPLICATIONS

This application claims the benefit under 35 U.S.C. §119(e) of U.S. Provisional Application Ser. No. 61/307,301, entitled “PATHOGEN SENSE AND DESTROY,” filed on Feb. 23, 2010, and U.S. Provisional Application Ser. No. 61/382,637, entitled “GENETICALLY PROGRAMMABLE PATHOGEN SENSE & DESTROY,” filed on Sep. 14, 2010, the disclosures of each of which are incorporated by reference herein in their entireties.

### GOVERNMENT INTEREST

This invention was made with Government support under Grant No. N00014-07-1-0069, awarded by the Office of Naval Research. The government has certain rights in this invention.

### FIELD OF THE INVENTION

The invention relates to compositions and methods for sensing and destroying specific pathogens.

### BACKGROUND OF THE INVENTION

Worldwide, nearly 2 million people per year die from diarrhea and other water-borne diseases, the vast majority of them children in Third World countries. Due to selective pressure, many of the pathogens responsible for these diseases have become resistant to “first-line” drugs and second- or third-line drugs can be much more expensive and potentially toxic.

Several enteric pathogens inhabit the lower gastrointestinal tract and cause localized disease following their acquisition through the fecal-oral route. For example, V. cholera, a motile Gram-negative human pathogen, results in a wide spectrum of diseases with various severities, including a fatality rate of approximately 50% if untreated (Nelson et al., (2009) Nature 7(10):693-702).

Shigella is a Gram-negative bacterium that is the principal agent of bacillary dysentery also called shigellosis. Three Shigella groups out of four are the major disease-causing species: S. flexneri is the most frequently isolated species worldwide and accounts for 60% of cases in the developing world; S. sonnei causes 77% of cases in the developed world, compared to only 15% of cases in the developing world; and S. dysenteriae is usually the cause of epidemics of dysentery. The serotype 1 of S. dysenteriae (Sd1) is of concern due to its expression of the Shiga toxin (Stx) which is cytotoxic, neurotoxic and enterotoxic. It is the cause of epidemic dysentery and can cause vicious outbreaks in confined populations. It targets glomerular epithelial cells, central nervous system and microvascular endothelial cells causing haemolytic-uremic syndrome (HUS) and seizures. A major obstacle to the control of Sd1 is its resistance to antimicrobial drugs.

### SUMMARY OF INVENTION

Overall, there is an urgent need to develop new anti-bacterial strategies. Described herein is a versatile and effective cellular sense-and-destroy system capable of adapting and responding to a large variety of target pathogens in multiple contexts. The system can function without human intervention, and may therefore be easily deployed in remote or access-compromised environments including, for example, contaminated water supply systems where such pathogens often fester. Compositions and methods described herein involve recombinant cells that are genetically programmed with sensors, information processing, and actuation. Significantly, methods described herein have widespread applications for pathogen control including targeting antibiotic-resistant microbial strains.

Aspects of the invention relate to cells that recombinantly expresses (1) a detection circuit, (2) optionally a signal amplifying circuit, and (3) a secretion circuit that secretes a factor that specifically recognizes and destroys a specific pathogen. In some embodiments the cell is a bacterial cell, such as a Gram negative bacterial cell. In certain embodiments, the cell is an Escherichia coli (E. coli) cell, such as an E. coli Nissle 1917 (EcN) cell. In some embodiments, the cell is an algal cell, a fungal cell (including a yeast cell), an insect cell or an animal cell. In certain embodiments, the cell is a mammalian cell such as a human cell. In some embodiments, the cell is a T cell or a B cell.

In some embodiments, the cell detects one or more molecules produced by one or more specific pathogens. In certain embodiments, one or more of the molecules produced by one or more specific pathogens is a quorum sensing molecule. In some embodiments, one or more of the specific pathogens is a bacterial pathogen, such as a Pseudomonas bacterial pathogen. In certain embodiments, the bacterial pathogen is a Pseudomonas aeruginosa bacterial pathogen.

In some embodiments, the cell detects 3-oxo-C12-homoserine lactone (3OC₁₂HSL) that is secreted by the Pseudomonas aeruginosa bacterial pathogen. The cell can comprise a transcriptional regulator, such as LasR, that regulates expression of one or more genes in response to 3OC₁₂HSL. In some embodiments, the signal amplifying circuit in the cell amplifies a response in the cell to the one or more specific pathogens. The signal amplifying circuit can comprise a transcriptional repressor downstream of a promoter that is regulated, such as the las promoter. In certain embodiments, the transcriptional repressor is CI. In certain embodiments, the signal amplifying circuit regulates production of a factor that is secreted by the recombinant cell to destroy the Pseudomonas aeruginosa bacterial pathogen.

In other embodiments, the bacterial pathogen is a Vibrio bacterial pathogen, such as a Vibrio cholerae bacterial pathogen. In certain embodiments, the cell detects CAI-1 that is secreted by the Vibrio cholerae bacterial pathogen. The cell can comprise a signal amplifying circuit that regulates production of a factor that is secreted by the cell to specifically destroy the Vibrio bacterial pathogen. In some embodiments, the cell expresses CqsS, LuxU and/or LuxO, which can be codon-optimized. In some embodiments, one or more of the bacterial pathogens is an antibiotic-resistant bacterial pathogen.

In some embodiments, the pathogen is a viral pathogen or a fungal pathogen. The pathogen can be present in a low cell density and can occur in vivo or in vitro. In some embodiments, the cell detects more than one specific pathogen.

In some embodiments, the factor that is secreted by the cell is an antimicrobial peptide. In certain embodiments, the factor that is secreted by the cell is a bacteriocin such as a lysin. In some embodiments, a flagellar gene, or a portion thereof, is expressed as a fusion with the lysin in the cell. The expression of the flagellar gene, or a portion thereof, fused to the lysin, can be transcriptionally regulated in the cell in response to detection of one or more specific pathogens. In certain embodiments, the flagellar gene is flgM or fliC. In some embodiments, the 5′UTR and/or the 3′UTR of the flagellar gene is fused to the lysin. In certain embodiments, the flagellar protein is linked to the lysin through a flexible linker.

In some embodiments, the lysin is a colicin or a pyocin. In certain embodiments, the colicin or pyocin comprises a chimeric recognition domain that is modified such that the colicin or pyocin specifically recognizes and destroys a specific pathogen. In some embodiments, the lysin is produced together with an immunity protein that protects the cell that secretes the lysin from being destroyed by the lysin. In certain embodiments, the factor that is secreted by the cell is a colicin that recombinantly expresses recognition and/or translocation domains, or portions thereof, of a pyocin and specifically destroys a P. aeruginosa pathogen.

In some embodiments, one or more flagellar genes in the cell, such as flagellar genes within the FliCDST operon, are deleted or mutated.

In some embodiments, the cell secretes a bacteriocin that is specific for V. cholerae. In certain embodiments, the bacteriocin is selected from the group consisting of Morricin 269, Kurstacin 287, Kenyacin 404, Entomocin 420 and Tolworthcin 524. In some embodiments, the cell produces a secreted factor by cell suicide. In certain embodiments, the secreted factor is a chemokine-derived antimicrobial peptide (CDAP).

In some embodiments, the bacterial pathogen is a Shigella bacterial pathogen or a Salmonella bacterial pathogen. In certain embodiments, the bacterial pathogen is a Shigella dysenteriae bacterial pathogen. In some embodiments, the cell detects AI-3 and its lambdoid phage. In certain embodiments, the cell expresses QseC and QseB. In some embodiments, the cell expresses molecular mimics of Shiga toxin receptors. In certain embodiments, the cell expresses a chimeric LPS in the outer membrane of the cell. In certain embodiments, the chimeric LPS contains a mutation in the waaO gene and/or the chimeric LPS terminates in Gal (α1, 4)Gal(β1, 4)(Glc).

In some embodiments, the cell expresses the Shigella lambdoid phage specific receptor, YaeT. In certain embodiments, the cell expresses Clustered Regularly Interspaced Short Palindromic Repeats (CRISPRs) sequences specific to incoming phage, and Shigella specific bacteriocin. In some embodiments, the Clustered Regularly Interspaced Short Palindromic Repeats (CRISPRs) sequences and the Shigella specific bacteriocin are expressed on a high copy number plasmid. In some embodiments, the Shigella specific bacteriocin is Colicin U. In certain embodiments, the receptor and translocase domains of Colicin U are fused to nuclease and immunity domains of E. coli Colicin E3.

In some embodiments, the cell is a component of a probiotic, a pharmaceutical preparation, a vaccine, a food or nutraceutical, a biospray and/or a water supply system.

Further aspects of the invention relate to methods for destroying a specific pathogen, the method comprising, providing a cell that recombinantly expresses (1) a detection circuit, (2) optionally a signal amplifying circuit, and (3) a secretion circuit that secretes a factor that specifically recognizes and destroys the specific pathogen.

In some embodiments the cell is a bacterial cell, such as a Gram negative bacterial cell. In certain embodiments, the cell is an Escherichia coli (E. coli) cell, such as an E. coli Nissle 1917 (EcN) cell. In some embodiments, the cell is an algal cell, a fungal cell (including a yeast cell), an insect cell or an animal cell. In certain embodiments, the cell is a mammalian cell such as a human cell. In some embodiments, the cell is a T cell or a B cell.

In some embodiments, the cell detects one or more molecules produced by one or more specific pathogens. In certain embodiments, one or more of the molecules produced by one or more specific pathogens is a quorum sensing molecule. In some embodiments, one or more of the specific pathogens is a bacterial pathogen, such as a Pseudomonas bacterial pathogen. In certain embodiments, the bacterial pathogen is a Pseudomonas aeruginosa bacterial pathogen.

In some embodiments, the cell detects 3-oxo-C12-homoserine lactone (3OC₁₂HSL) that is secreted by the Pseudomonas aeruginosa bacterial pathogen. The cell can comprise a transcriptional regulator, such as LasR, that regulates expression of one or more genes in response to 3OC₁₂HSL. In some embodiments, the signal amplifying circuit in the cell amplifies a response in the cell to the one or more specific pathogens. The signal amplifying circuit can comprise a transcriptional repressor downstream of a promoter that is regulated, such as the las promoter. In certain embodiments, the transcriptional repressor is CI. In certain embodiments, the signal amplifying circuit regulates production of a factor that is secreted by the recombinant cell to destroy the Pseudomonas aeruginosa bacterial pathogen.

In other embodiments, the bacterial pathogen is a Vibrio bacterial pathogen, such as a Vibrio cholerae bacterial pathogen. In certain embodiments, the cell detects CAI-1 that is secreted by the Vibrio cholerae bacterial pathogen. The cell can comprise a signal amplifying circuit that regulates production of a factor that is secreted by the cell to specifically destroy the Vibrio bacterial pathogen. In some embodiments, the cell expresses CqsS, LuxU and/or LuxO, which can be codon-optimized. In some embodiments, one or more of the bacterial pathogens is an antibiotic-resistant bacterial pathogen.

In some embodiments, the pathogen is a viral pathogen or a fungal pathogen. The pathogen can be present in a low cell density and can occur in vivo or in vitro. In some embodiments, the cell detects more than one specific pathogen.

In some embodiments, the factor that is secreted by the cell is an antimicrobial peptide. In certain embodiments, the factor that is secreted by the cell is a bacteriocin such as a lysin. In some embodiments, a flagellar gene, or a portion thereof, is expressed as a fusion with the lysin in the cell. The expression of the flagellar gene, or a portion thereof, fused to the lysin, can be transcriptionally regulated in the cell in response to detection of one or more specific pathogens. In certain embodiments, the flagellar gene is flgM or fliC. In some embodiments, the 5′UTR and/or the 3′UTR of the flagellar gene is fused to the lysin. In certain embodiments, the flagellar protein is linked to the lysin through a flexible linker.

In some embodiments, the lysin is a colicin or a pyocin. In certain embodiments, the colicin or pyocin comprises a chimeric recognition domain that is modified such that the colicin or pyocin specifically recognizes and destroys a specific pathogen. In some embodiments, the lysin is produced together with an immunity protein that protects the cell that secretes the lysin from being destroyed by the lysin. In certain embodiments, the factor that is secreted by the cell is a colicin that recombinantly expresses recognition and/or translocation domains, or portions thereof, of a pyocin and specifically destroys a P. aeruginosa pathogen.

In some embodiments, one or more flagellar genes in the cell, such as flagellar genes within the FliCDST operon, are deleted or mutated.

In some embodiments, the cell secretes a bacteriocin that is specific for V. cholerae. In certain embodiments, the bacteriocin is selected from the group consisting of Morricin 269, Kurstacin 287, Kenyacin 404, Entomocin 420 and Tolworthcin 524. In some embodiments, the cell produces a secreted factor by cell suicide. In certain embodiments, the secreted factor is a chemokine-derived antimicrobial peptide (CDAP).

In some embodiments, the bacterial pathogen is a Shigella bacterial pathogen or a Salmonella bacterial pathogen. In certain embodiments, the bacterial pathogen is a Shigella dysenteriae bacterial pathogen. In some embodiments, the cell detects AI-3 and its lambdoid phage. In certain embodiments, the cell expresses QseC and QseB. In some embodiments, the cell expresses molecular mimics of Shiga toxin receptors. In certain embodiments, the cell expresses a chimeric LPS in the outer membrane of the cell. In certain embodiments, the chimeric LPS contains a mutation in the waaO gene and/or the chimeric LPS terminates in Gal (α1, 4)Gal(β1, 4)(Glc).

In some embodiments, the cell expresses the Shigella lambdoid phage specific receptor, YaeT. In certain embodiments, the cell expresses Clustered Regularly Interspaced Short Palindromic Repeats (CRISPRs) sequences specific to incoming phage, and Shigella specific bacteriocin. In some embodiments, the Clustered Regularly Interspaced Short Palindromic Repeats (CRISPRs) sequences and the Shigella specific bacteriocin are expressed on a high copy number plasmid. In some embodiments, the Shigella specific bacteriocin is Colicin U. In certain embodiments, the receptor and translocase domains of Colicin U are fused to nuclease and immunity domains of E. coli Colicin E3.

In some embodiments, the cell is a component of a probiotic, a pharmaceutical preparation, a vaccine, a food or nutraceutical, a biospray and/or a water supply system.

In some embodiments, the method is a method of treating or preventing a disease, such as an infectious disease. In some embodiments, the cell is provided in a therapeutically effective amount to a subject in need thereof. In certain embodiments, the pathogen is within the gastrointestinal tract and/or the lung of a subject. In some embodiments, the method is a method of sterilization, such as sterilization of medical equipment.

Further aspects of the invention relate to methods for protecting against a specific pathogen, the method comprising, administering to a subject in need thereof an effective amount of a composition comprising a cell that recombinantly expresses (1) a detection circuit, (2) optionally a signal amplifying circuit, and (3) a secretion circuit that secretes a factor that specifically recognizes and destroys the specific pathogen.

In some embodiments the cell is a bacterial cell, such as a Gram negative bacterial cell. In certain embodiments, the cell is an Escherichia coli (E. coli) cell, such as an E. coli Nissle 1917 (EcN) cell. In some embodiments, the cell is an algal cell, a fungal cell (including a yeast cell), an insect cell or an animal cell. In certain embodiments, the cell is a mammalian cell such as a human cell. In some embodiments, the cell is a T cell or a B cell.

In some embodiments, the cell detects one or more molecules produced by one or more specific pathogens. In certain embodiments, one or more of the molecules produced by one or more specific pathogens is a quorum sensing molecule. In some embodiments, one or more of the specific pathogens is a bacterial pathogen, such as a Pseudomonas bacterial pathogen. In certain embodiments, the bacterial pathogen is a Pseudomonas aeruginosa bacterial pathogen.

In some embodiments, the cell detects 3-oxo-C12-homoserine lactone (3OC₁₂HSL) that is secreted by the Pseudomonas aeruginosa bacterial pathogen. The cell can comprise a transcriptional regulator, such as LasR, that regulates expression of one or more genes in response to 3OC₁₂HSL. In some embodiments, the signal amplifying circuit in the cell amplifies a response in the cell to the one or more specific pathogens. The signal amplifying circuit can comprise a transcriptional repressor downstream of a promoter that is regulated, such as the las promoter. In certain embodiments, the transcriptional repressor is CI. In certain embodiments, the signal amplifying circuit regulates production of a factor that is secreted by the recombinant cell to destroy the Pseudomonas aeruginosa bacterial pathogen.

In other embodiments, the bacterial pathogen is a Vibrio bacterial pathogen, such as a Vibrio cholerae bacterial pathogen. In certain embodiments, the cell detects CAI-1 that is secreted by the Vibrio cholerae bacterial pathogen. The cell can comprise a signal amplifying circuit that regulates production of a factor that is secreted by the cell to specifically destroy the Vibrio bacterial pathogen. In some embodiments, the cell expresses CqsS, LuxU and/or LuxO, which can be codon-optimized. In some embodiments, one or more of the bacterial pathogens is an antibiotic-resistant bacterial pathogen.

In some embodiments, the pathogen is a viral pathogen or a fungal pathogen. The pathogen can be present in a low cell density and can occur in vivo or in vitro. In some embodiments, the cell detects more than one specific pathogen.

In some embodiments, the factor that is secreted by the cell is an antimicrobial peptide. In certain embodiments, the factor that is secreted by the cell is a bacteriocin such as a lysin. In some embodiments, a flagellar gene, or a portion thereof, is expressed as a fusion with the lysin in the cell. The expression of the flagellar gene, or a portion thereof, fused to the lysin, can be transcriptionally regulated in the cell in response to detection of one or more specific pathogens.

In certain embodiments, the flagellar gene is flgM or fliC. In some embodiments, the 5′UTR and/or the 3′UTR of the flagellar gene is fused to the lysin. In certain embodiments, the flagellar protein is linked to the lysin through a flexible linker.

In some embodiments, the lysin is a colicin or a pyocin. In certain embodiments, the colicin or pyocin comprises a chimeric recognition domain that is modified such that the colicin or pyocin specifically recognizes and destroys a specific pathogen. In some embodiments, the lysin is produced together with an immunity protein that protects the cell that secretes the lysin from being destroyed by the lysin. In certain embodiments, the factor that is secreted by the cell is a colicin that recombinantly expresses recognition and/or translocation domains, or portions thereof, of a pyocin and specifically destroys a P. aeruginosa pathogen.

In some embodiments, one or more flagellar genes in the cell, such as flagellar genes within the FliCDST operon, are deleted or mutated.

In some embodiments, the cell secretes a bacteriocin that is specific for V. cholerae. In certain embodiments, the bacteriocin is selected from the group consisting of Morricin 269, Kurstacin 287, Kenyacin 404, Entomocin 420 and Tolworthcin 524. In some embodiments, the cell produces a secreted factor by cell suicide. In certain embodiments, the secreted factor is a chemokine-derived antimicrobial peptide (CDAP).

In some embodiments, the bacterial pathogen is a Shigella bacterial pathogen or a Salmonella bacterial pathogen. In certain embodiments, the bacterial pathogen is a Shigella dysenteriae bacterial pathogen. In some embodiments, the cell detects AI-3 and its lambdoid phage. In certain embodiments, the cell expresses QseC and QseB. In some embodiments, the cell expresses molecular mimics of Shiga toxin receptors. In certain embodiments, the cell expresses a chimeric LPS in the outer membrane of the cell. In certain embodiments, the chimeric LPS contains a mutation in the waaO gene and/or the chimeric LPS terminates in Gal (α1, 4)Gal(β1, 4)(Glc).

In some embodiments, the cell expresses the Shigella lambdoid phage specific receptor, YaeT. In certain embodiments, the cell expresses Clustered Regularly Interspaced Short Palindromic Repeats (CRISPRs) sequences specific to incoming phage, and Shigella specific bacteriocin. In some embodiments, the Clustered Regularly Interspaced Short Palindromic Repeats (CRISPRs) sequences and the Shigella specific bacteriocin are expressed on a high copy number plasmid. In some embodiments, the Shigella specific bacteriocin is Colicin U. In certain embodiments, the receptor and translocase domains of Colicin U are fused to nuclease and immunity domains of E. coli Colicin E3.

In some embodiments, the cell is a component of a probiotic, a pharmaceutical preparation, a vaccine, a food or nutraceutical, a biospray and/or a water supply system.

In some embodiments, the method is a method of treating or preventing a disease, such as an infectious disease. In some embodiments, the cell is provided in a therapeutically effective amount to a subject in need thereof. In certain embodiments, the pathogen is within the gastrointestinal tract and/or the lung of a subject. In some embodiments, the method is a method of sterilization, such as sterilization of medical equipment.

These and other aspects of the invention, as well as various embodiments thereof, will become more apparent in reference to the drawings and detailed description of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts an example of the cellular sense-and-destroy system. FIG. 1A presents a schematic of exemplary engineered pathways for detection and destruction of pathogens. FIG. 1B presents a graph showing a response curve. FIG. 1C presents a graph indicating differential killing by sentinel cells.

FIG. 2 depicts Pseudomonas aeruginosa (P. aeruginosa) signal detection. FIG. 2A presents a schematic of a genetic circuit for Las sentinel. FIG. 2B presents a graph showing 3OC₁₂HSL response curves for Las sentinel. FIG. 2C presents a graph showing receiver fluorescence as a function of pathogen density.

FIG. 3 presents a schematic depicting coupling of flagellar gene regulation to flagellum assembly (adapted from Chevance, F. F. V. et al., Nature Reviews Microbiology, 2009).

FIG. 4 presents a schematic representation of bacteriocin killing. FIG. 4A shows the natural E. coli Colicin intra-species killing mechanism. FIG. 4B shows an embodiment of the engineered CoPy interspecies killing mechanism described herein. T=translocase domain; R=recognition domain; N=nuclease domain; I=immunity domain.

FIG. 5 depicts engineering of a chimeric killer protein. The translocation and recognition domain of Colicin are replaced by the corresponding domains from Pyocin. The resultant chimeric protein has altered functionality and specificity. T=translocase domain; R=recognition domain; N=nuclease domain; I=immunity domain.

FIG. 6 demonstrates the specificity of sense-and-destroy systems described herein. FIG. 6A presents a graph demonstrating that growth of E. coli sentinels is unaffected by purified CoPy (84.7 m.w., 1 ug=11.793 picomoles (pm), conc.=163 ng/ul, 1 ul=1.92 pm). FIG. 6B presents a graph demonstrating that growth of PAO-1 is completely inhibited by purified CoPy from the cell lysate.

FIG. 7 demonstrates the FlgM-CoPy fusion and the effect of the purified fusion protein on PAO-1. FIG. 7A depicts a Western Blot showing the secretion of the FlgM-CoPy fusion. (IN represents cell lysate, OUT represents filter sterilized supernatant). FIG. 7B demonstrates that growth of PAO-1 is inhibited by purified His-FlgM-CoPy (92.521 m.w, 1 ug=10.81 pm), conc.=320 ng/ul, 1 ul=3.4 pm).

FIG. 8 reveals the effect of exogenous FlgM-CoPy on PAO-1. FIG. 8A shows PAO-1 growth/death due to exogenous FlgM-CoPy. FIG. 8B depicts microscopic images (10×) of negative control and sentinel cells (ECN) co-cultured with PAO-1 with a seeding ratio of 10:1.

FIG. 9 presents a schematic of the V. cholerae quorum-sensing circuit (adapted from Wingreen and Levin (2006) PLOS Biology 4(9):e299.

FIG. 10 presents a schematic of an embodiment of the V. cholerae sense-and-destroy system. FIG. 10A depicts sentinel activity when V. cholerae pathogen is at low cell density. FIG. 10B depicts expression and secretion of killer protein from the sentinels at high pathogen cell density.

FIG. 11 depicts PAO-1 C₄HSL signal amplification. FIG. 11A presents a schematic of a genetic circuit for PAO-1 C₄HSL signal amplification. FIG. 11B presents log phase responses of several different qsc promoters to C4HSL. FIG. 11C presents log phase C₄HSL responses of several different combinations of signal amplifiers coupled to the qsc promoters. The notation “mut x” indicates the mutant version of λ_{P(R)} we used. Note the dramatic signal amplification achieved with mut0/qsc119, mut5/qsc131, and mut6/qsc131.

FIG. 12 presents a summary of several examples of pathogen sense-and-destroy systems.

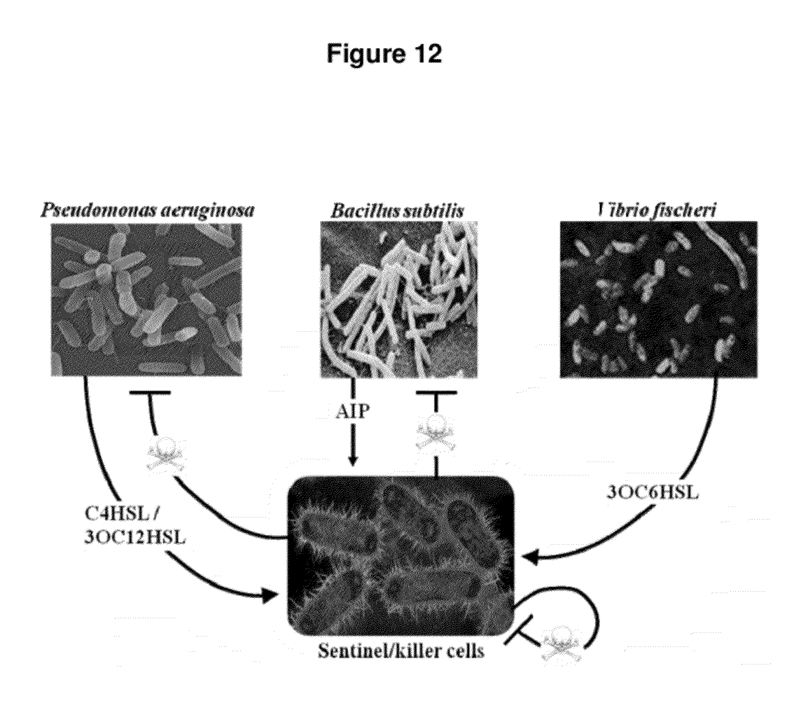

FIG. 13 summarizes FliC based secretion. FIG. 13A depicts a schematic of flagellar proteins. FIG. 13B depicts a schematic of engineering flagellar gene expression (from Chevance et al., Nature Reviews, 2008).

FIG. 14 presents results from plate reader data with E. coli and PAO1. FIG. 14A demonstrates P. aeruginosa optical density. FIG. 14B demonstrates cell density in E. coli and PAO1 lysates. FIG. 14C demonstrates cell density of E. coli cells incubated with purified CoPy. FIG. 14D demonstrates cell density in PAO1 cells incubated with purified CoPy.

FIG. 15 depicts system architecture of S. dysenteriae sense-and-destroy. 1) Sentinels detect AI-3; 2) QseB initiates transcription from P_{AI-3}; 3) Receptors for the Shiga toxin and Stx phage are expressed; 4) Phage binds to its specific receptor and inserts its DNA; 5) Sentinels detect incoming phage; 6) The phage is destroyed by CRISPRs and Shigella specific colicin is secreted to kill the pathogen.

FIG. 16 presents schematics related to the Shigella sense-and-destroy. FIG. 16A depicts AI-3 signaling in Shigella and Salmonella. FIG. 16B depicts organization and regulation of phage genes.

FIG. 17 presents a schematic depicting the mechanism of CRISPR.

### DETAILED DESCRIPTION

Aspects of the invention relate to an anti-microbial “sense-and-destroy” system created by engineering cells to detect the presence of a pathogen and secrete specific factors that destroy the pathogen. The system can analyze environmental conditions and execute an “intelligent” response by utilizing multiple, customized treatments. This approach offers a variety of advantages over previous approaches for destroying pathogens. It presents a single, integrated solution to eradicating multiple threats with a method that is a rapid, selective, and highly sensitive. Additionally, this system will detect and kill a pathogen at low densities, eliminating the problem at its source. This application-driven system is highly specific for pathogen detection and destruction. Changes in identity and abundance of an input signal molecule, indicating the appearance or disappearance of certain pathogens, elicit changes in output and select different specific factors to express, based on the identity of the threat. Thus, the cells comprise a synthetic defense system that can monitor the progression of a chosen treatment and alter the type of treatment on demand. Since only small doses of the antimicrobial factors are needed, this approach also involves less harmful side effects than other antibiotics. Additionally, this strategy is effective against antibiotic-resistant pathogen strains.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Cells described herein specifically detect the presence of one or more pathogens. In some embodiments, the cell detects the presence of the one or more pathogens through detection of one or more molecules, such as diffusible molecules, that are produced by the one or more pathogens. In some embodiments, a molecule produced by a specific pathogen is a quorum sensing molecule. As used herein, quorum sensing refers to the use of population density to coordinate certain cellular behaviors. A quorum sensing molecule refers to a molecule produced by a cell that can signal population density of the population of cells containing that cell. In some embodiments, the cell detects the presence of the one or more pathogens through detection of a protein. It should be appreciated that aspects of the invention encompass any mechanism by which a cell can detect the presence of a pathogen.

Cells associated with the invention express genetic circuits. As used herein, a genetic circuit refers to a collection of recombinant genetic components that responds to one or more input molecules and performs a specific function, such as the regulation of the expression of one or more genes. Specifically, a detection circuit refers to a collection of recombinant genetic components in a cell that is responsive to one or more input molecules produced by a pathogen and that regulates the expression of one or more genes in response to detection of the pathogen. The one or more input molecules produced by the pathogen can be quorum sensing molecules. In some embodiments, a quorum sensing molecule is an oligopeptide, an N-Acyl homoserine lactone (AHL) an autoinducer or a pheromone.

In some embodiments, the detection circuit responds to one type of molecule produced by one type of pathogen. In other embodiments, the detection circuit responds to more than one type of molecule produced by one or more different types of pathogens. In some embodiments, a cell expresses one type of detection circuit, while in other embodiments, a cell expresses multiple different types of detection circuits that respond to different molecules produced by one or more different types of pathogens.

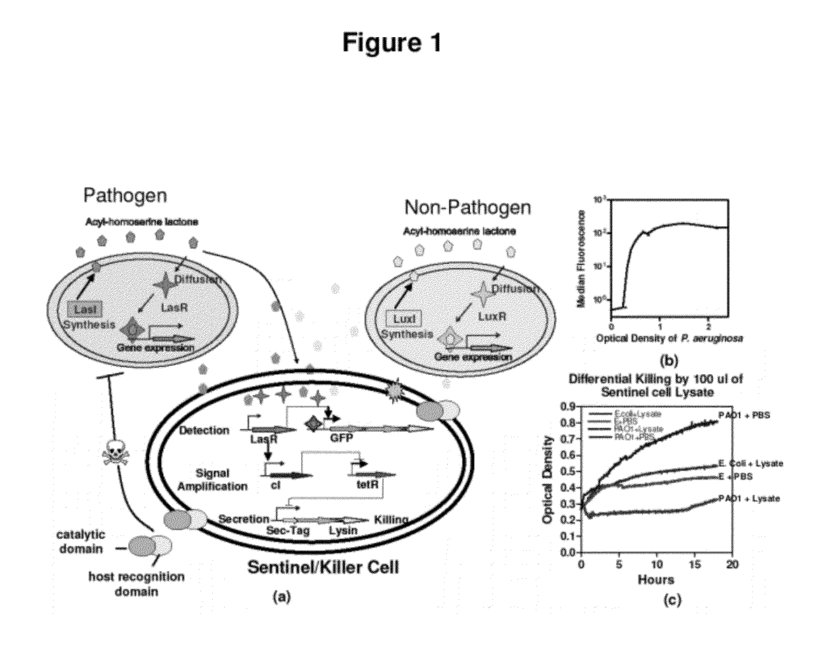

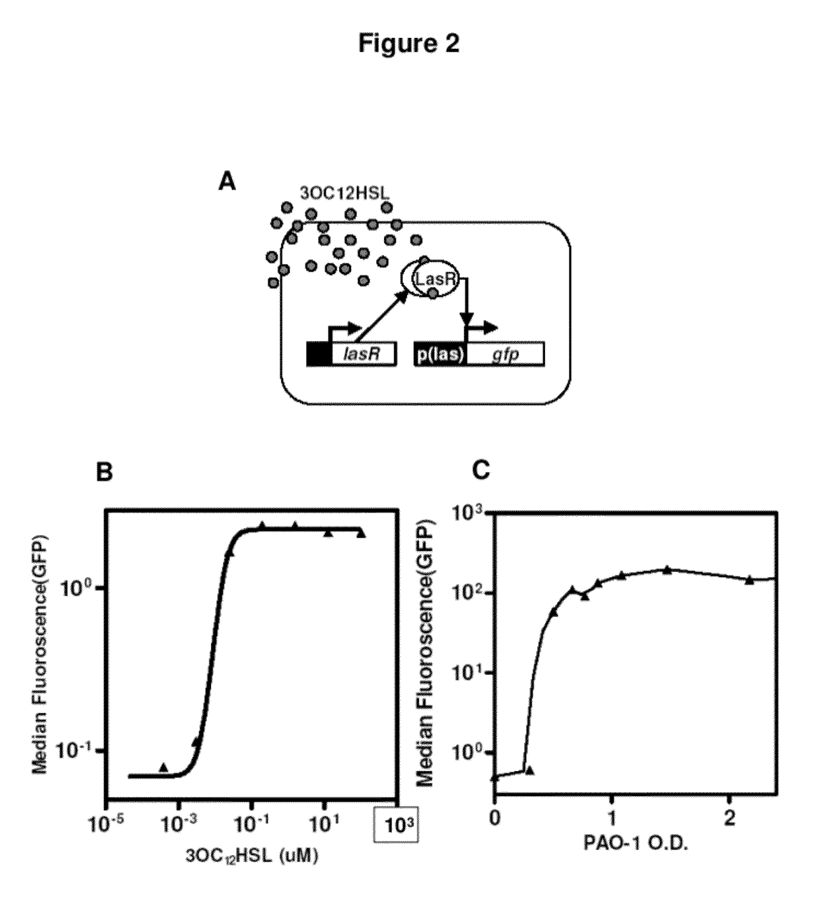

Several exemplary detection circuits are described and demonstrated in the Examples section. Example 1 demonstrates a sense-and-destroy system designed to target P. aeruginosa cells. P. aeruginosa cells produce acylated homoserine lactone (AHL) autoinducers that diffuse freely between the cytoplasm and the environment and interact directly with the transcriptional regulator LasR. In some embodiments of the sense-and-destroy system, such as that described in Example 1, an E. coli sentinel/killer cell is engineered to express a detection circuit that contains LasR. The detection circuit, through LasR, responds to the AHL autoinducer 3OC₁₂HSL, produced by the P. aeruginosa cells, and LasR regulates expression of downstream genes. For example, FIG. 1 and FIG. 2A demonstrate a detection circuit wherein LasR regulates expression of GFP, allowing verification of successful detection by the detection circuit within the sense-and-destroy system.

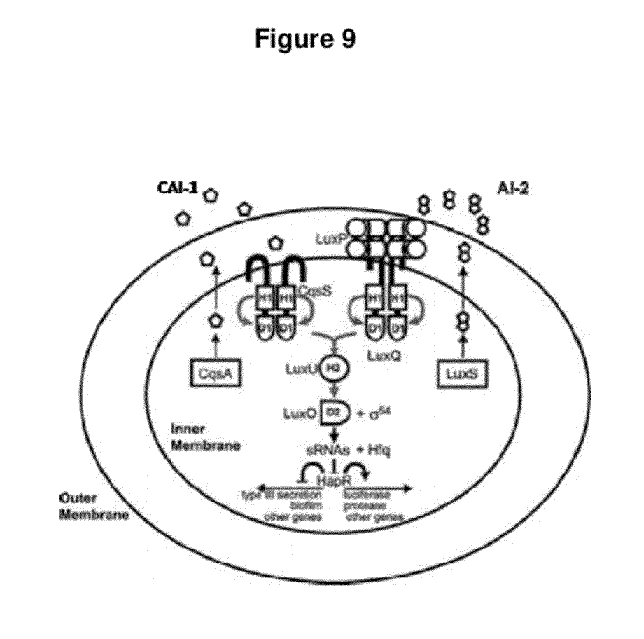

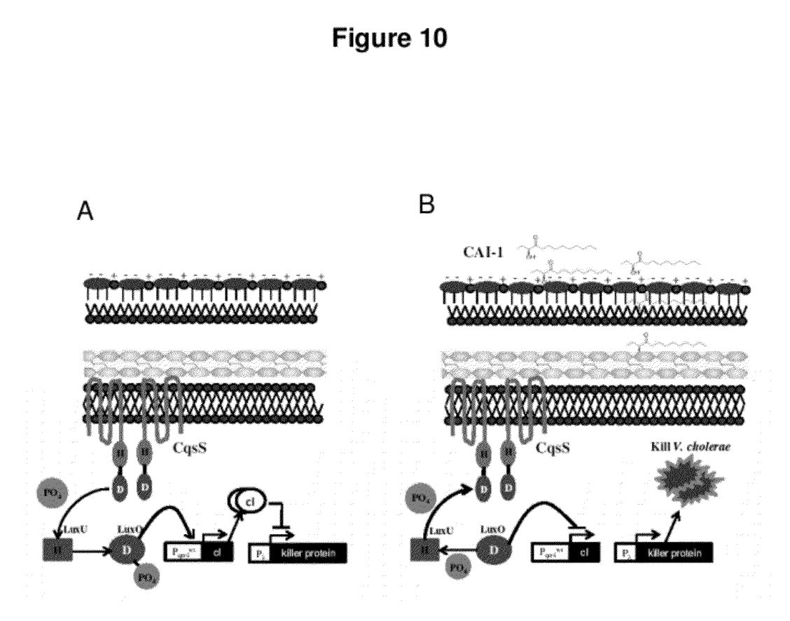

Example 2 describes a sense-and-destroy system for targeting the V. cholerae pathogen. FIG. 9 demonstrates the V. cholerae quorum sensing circuit which involves a set of quorum sensing molecules called AI-2, including the molecule CAI-1 ((S)-3-hydroxytridecan-4-one). In embodiments such as that described in Example 2, the sense-and-destroy cell is engineered to express a detection circuit that responds to CAI-1. The detection circuit, demonstrated in FIG. 10, expresses codon-optimized versions of CqsS, LuxU and LuxO. The detection circuit responds to the presence of the quorum sensing molecule CAI-1, produced by V. cholerae cells, and LuxO, within the detection circuit, regulates the expression of downstream genes in response to the presence of the pathogen.

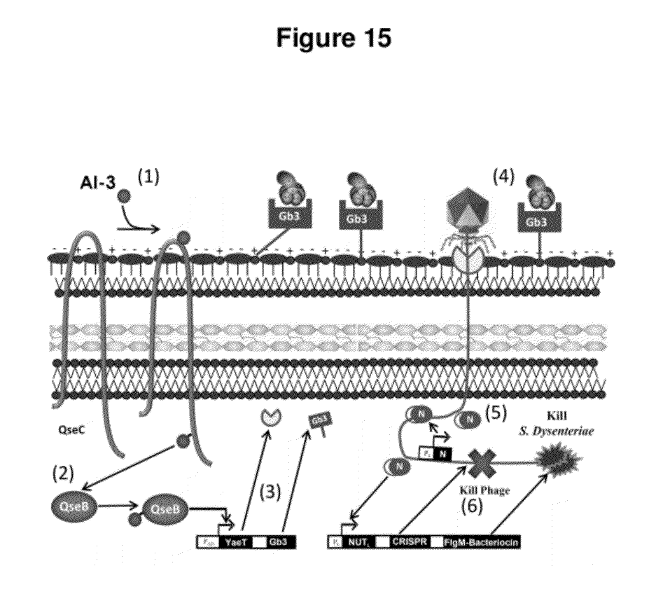

Example 4 describes a sense-and-destroy system for targeting Shigella pathogens (FIG. 15). In embodiments such as that described in Example 4, the sentinel/killer cell responds to the quorum sensing signal autoinducer AI-3, along with its lambdoid phage, produced by the Shigella pathogen. The sentinel/killer cells are engineered to express a detection circuit that comprises the transmembrane histidine kinase (HK) QseC which detects AI-3, and the response regulator QseB.

It should be appreciated that the examples of detection circuits described herein are non-limiting. One of ordinary skill in the art would understand based on the teachings herein and the knowledge in the art of regulation of gene expression, how to design detection circuits to respond to a wide range of molecules produced by a wide range of pathogens. In some embodiments, the detection circuit comprises one or more transcriptional activators.

Cells associated with the invention optionally also express a signal amplifying circuit. As used herein, a signal amplifying circuit refers to a collection of recombinant genetic components that responds to an input signal and amplifies the effect of the input signal. Placing a signal amplifying circuit downstream of a detection circuit within a sentinel/killer cell allows a cell to amplify the response produced by detection of a molecule such as a quorum sensing molecule.

In some embodiments, a signal amplifying circuit comprises one or more transcriptional activators and/or one or more transcriptional repressors. The transcriptional activators and transcriptional repressors can be expressed under the control of regulatable promoters. For example, in embodiments such as that described in Example 1, the transcriptional repressor CI is expressed under the control of the las promoter which is regulated in response to detection of the quorum sensing molecule. CI in turn regulates the expression of downstream genes. Since CI is an efficient transcriptional repressor, even small increases in CI levels can yield large changes in downstream gene expression. Similarly, in the embodiment described in Example 2, the signal amplifying circuit comprises a destabilized lambda repressor (cI-lva) expressed under a regulatable promoter, P_{qrr4wt}. This construct functions as a signal amplifier, downstream of the detection circuit, allowing the sentinel/killer cell to respond to even minute concentrations of the quorum sensing molecule when the pathogen is still in the early stages of infection.

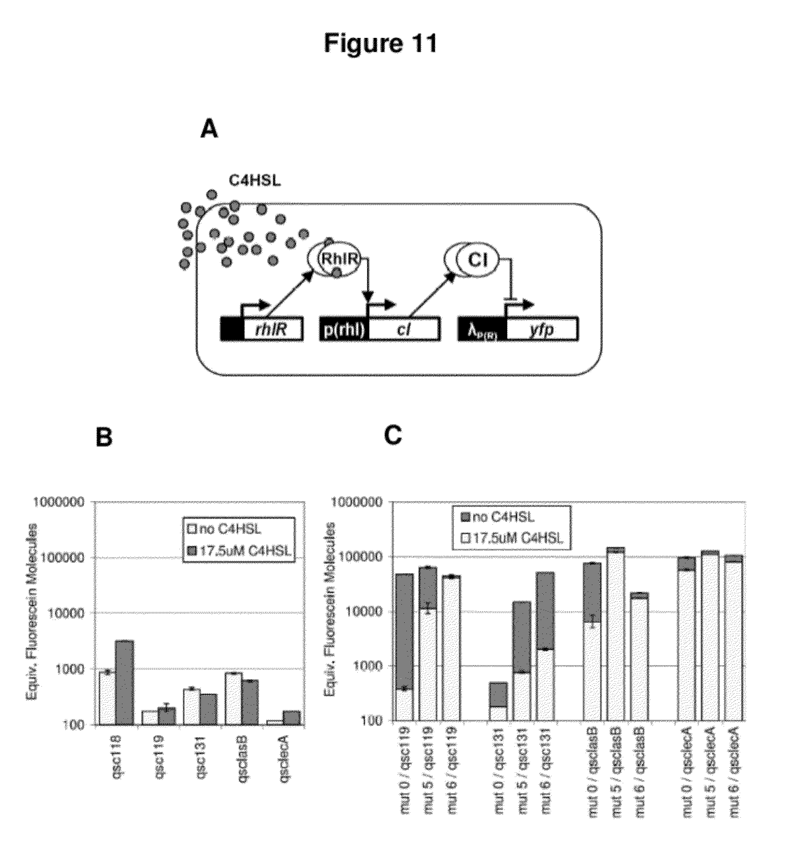

FIG. 11 demonstrates an example of a signal amplifying circuit of a PAO-1 C₄HSL quorum sensing signal (Karig and Weiss, 2005). In this example, the circuit amplifies a response to C₄HSL by expressing the transcriptional repressor CI downstream of a rhl promoter and placing λ_{P(R)} upstream of an output fluorescent protein. Since CI is a very efficient transcriptional repressor, even small increases in CI levels yield large changes in λ_{P(R)} activity, ultimately resulting in significant changes in final output signal, demonstrating the effectiveness of signal amplifying circuits.

It should be appreciated that the examples of signal amplifying circuits described herein are non-limiting. One of ordinary skill in the art would understand based on the teachings herein and the knowledge in the art of regulation of gene expression, how to design signal amplifying circuits to amplify responses to a wide range of molecules produced by a wide range of pathogens.

Cells associated with the invention also express secretion circuits. As used herein, a secretion circuit refers to a collection of recombinant genetic components that, in response to the presence of a pathogen, regulates secretion of factors that specifically recognize and destroy the pathogen. In sense-and-destroy systems described herein, secretion circuits are expressed downstream of the detection circuit and/or the signal amplifying circuit. In some embodiments, the detection circuit and/or the signal amplifying circuit regulates production of the factor that is secreted by the cell to destroy the pathogen.

In some embodiments, the secretion mechanism in a sense-and-destroy system makes use of the flagellar secretion apparatus in a cell, such as the flagellar type III secretion apparatus. For example, the secreted factor can be expressed as a fusion with a flagellar gene, or a portion thereof. The flagellar gene can be any flagellar gene in the cell. In some embodiments, the flagellar gene is flgM or fliC. In certain embodiments, the 5′UTR and/or the 3′UTR of a flagellar gene is fused to the secreted factor. In some embodiments, the flagellar protein is linked to the secreted factor through a flexible linker.

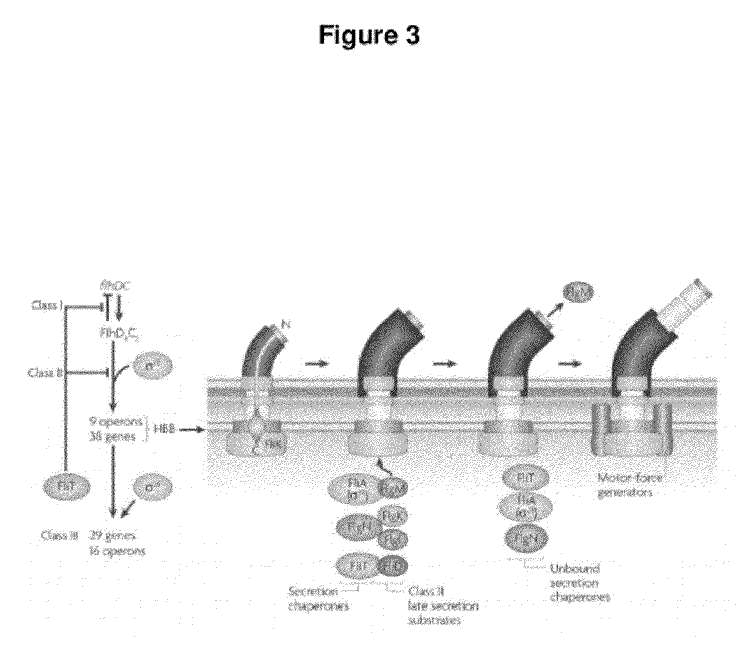

FIG. 3 presents a schematic of flagellar gene regulation and flagellum assembly in Salmonella typhimurium. Other types of bacteria, such as E. coli, have similar flagellar control. The expression of one or more of the flagellar genes can be transcriptionally regulated in response to detection of one or more specific pathogens by placing expression of the one or more flagellar genes under the control of a promoter that is expressed only when the pathogen is detected.

Aspects of the invention relate to the secretion of factors that recognize and destroy specific pathogens. In some embodiments, the secreted factor is a bacteriocin. As used herein, a bacteriocin refers to a proteinaceous toxin produced by bacteria to inhibit the growth of other bacterial strains. All type of bacteriocins are encompassed by aspects of the invention. Class I bacteriocins include small peptide inhibitors such as nisin. Class II bacteriocins include heat-stable proteins. Class IIa bacteriocins are characterized by possession of the N-terminal sequence: Tyr-Gly-Asn-Gly-Val-Xaa-Cys (SEQ ID NO:1). Class IIb bacteriocins are two-peptide bacteriocins while class IIc bacteriocins are circular bacteriocins. Class III bacteriocins are heat-labile proteins.

In some embodiments, the bacteriocin is a colicin or a pyocin, which are naturally expressed lysins in Gram-negative bacteria. Colicins and pyocins usually have three distinct domains: a recognition domain which binds specific receptors on the surface of the target species, a translocase domain which translocates the nuclease domain into the cell, and a nuclease domain which can be DNase or RNase and which kills a cell by cleaving its DNA or RNA.

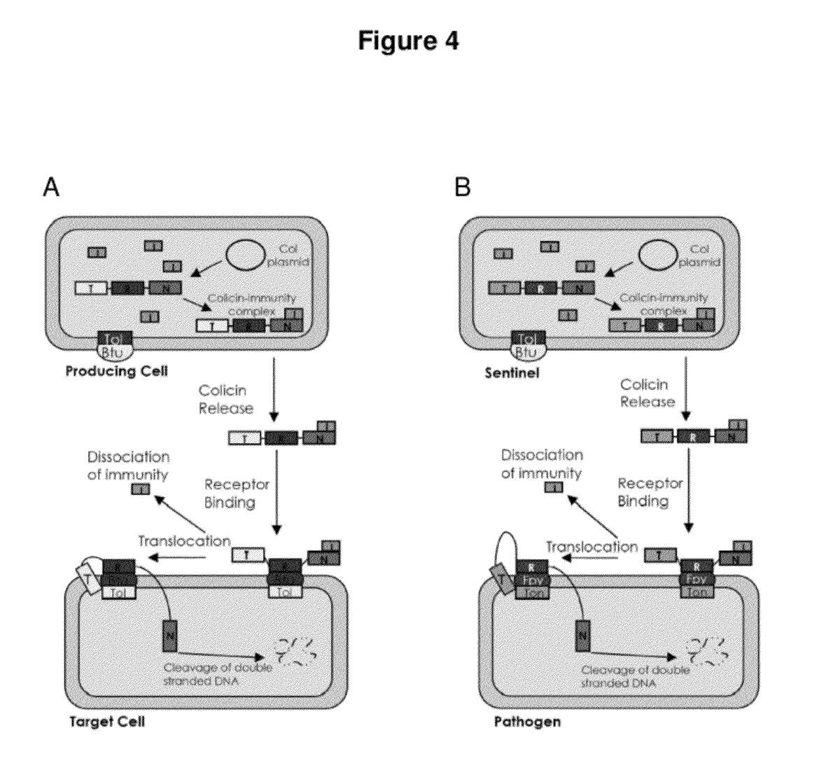

The cell that secretes the colicin is protected from the killing activity of the colicin because the colicin is produced along with an immunity protein to which it is translationally coupled and which forms a tight complex with the nuclease domain. In a target cell, the complex dissociates when the receptor domain binds the corresponding receptor on the target cell. Within the cytoplasm of the producing cell, nuclease domains are inactivated because they are bound by immunity proteins present in the cytoplasm. In some embodiments, target cells do not express the immunity protein and thus are sensitive to the colicin activity. FIG. 4 shows a schematic representation of the natural colicin killing mechanism. Pyocins are P. aeruginosa bacteriocins and have the same general structure as colicins.

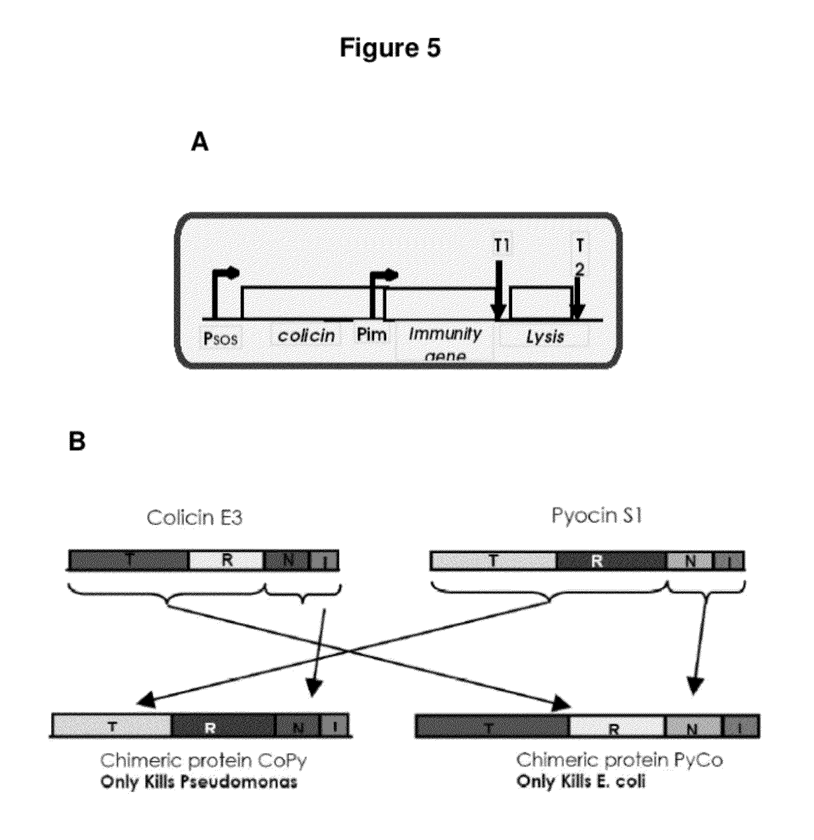

Aspects of the invention relate to novel bacteriocins in which one or more domains have been engineered to recognize and destroy specific pathogens. For example, in embodiments such as that described in Example 1, wherein a sense-and-destroy system within E. coli cells recognizes and destroys P. aeruginosa cells, a novel bacteriocin was created replacing the recognition and translocation domain of the colicin with that of a pyocin. The novel bacteriocin is termed herein “CoPy” due to its hybrid structure (FIG. 4B and FIG. 5). The novel bacteriocin, CoPy, kills only the P. aeruginosa pathogen while the E. coli sentinel/killer cell is unaffected by the protein due to the presence of the immunity protein in the E. coli cell and also due to the differential cell surface receptors. Novel bacteriocins, such as CoPy, can be expressed under a promoter that is regulated in response to detection of the pathogen.

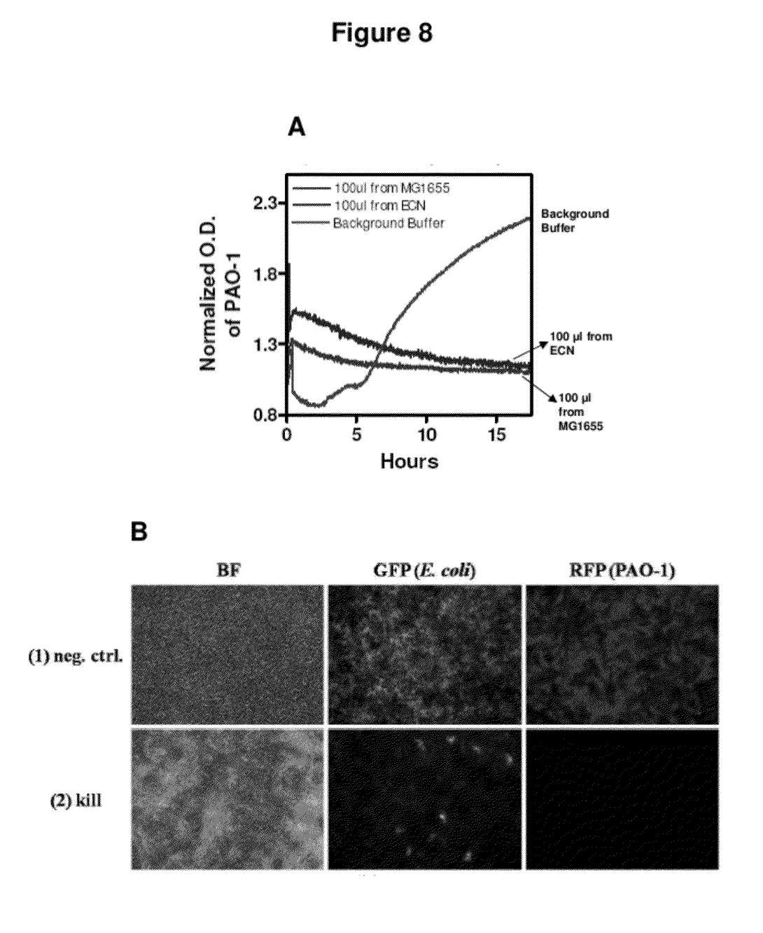

In embodiments such as that described in Example 1, the CoPy protein is fused to the flagellar protein FlgM and placed under the transcriptional control of the las promoter, which is active only when the pathogen is present. Co-culture of the E. coli (EcN) cells that express the sense-and-destroy system and P. aeruginosa (PAO-1) cells revealed that CoPy was successful in specifically destroying the PAO-1 cells (FIG. 8).

It should be appreciated that Example 1 represents a non-limiting embodiment and that the approach described herein for generating chimeric novel bacteriocins that recognize specific pathogens can be extended to other cell types and other pathogens, as one of ordinary skill in the art would recognize.

Example 2 describes engineering a bacteriocin specific for targeting the pathogen V. cholerae. In some embodiments, the bacteriocin is a bacteriocin that is synthesized in a Gram-positive soil bacterium such as Bacillus thuringiensis. Several non-limiting examples of such bacteriocins include Morricin 269, Kurstacin 287, Kenyacin 404, Entomocin 420 and Tolworthcin 524. These bacteriocins have been reported to selectively kill V. cholerae and are not effective against several other Gram-negative bacteria.

In some embodiments, a bacteriocin for targeting a specific pathogen is selected based, at least in part, on its specificity for targeting that pathogen. In some embodiments, other factors that are considered in selecting a bacteriocin for targeting a specific pathogen include: thermostability, resistance to α-amylase activity, resistance to RNase activity, resistance to lysozyme activity, activity at low and/or high pH, a preferred molecular mass range such as a molecular mass of approximately 10-25 kDa, and few or no cysteine residues.

Example 4 describes a sense-and-destroy system designed to target a Shigella pathogen such as S. dysenteriae. This example can be readily modified to detect and kill Salmonella and other related pathogens. In some embodiments, the pathogen is Salmonella enterica enterica, serovar Typhi. As described above, in some embodiments, the sentinel/killer cell detects AI-3 that is produced by the Shigella pathogen. In some embodiments, the sentinel/killer cell carries a mutation in the luxS gene, making it defective in producing AI-3 itself. In some embodiments, detection of AI-3 is not sufficient proof of the existence of a Shigella pathogen so the sentinel/killer cells employ a two-pronged approach to more specifically detect and destroy the pathogen with minimum damage to other cells, as described further below.

In some embodiments, the sentinel/killer cells express molecular mimics of the Shiga toxin (Stx1/Stx2) receptors (Gb3) on the surface of the cell. This allows for sequestration of the toxin, which is present if the pathogen is present, into the lumen of the cell. In some embodiments, the toxins are Stx1 variants (Stx1 and Stx1c), Stx2 variants (Stx2, Stx2c, Stx2d, Stx2e, and Stx2f) or variants of both in a range of combinations. Gb3 (Saccharide structure: Gal (α1, 4) Gal (β1, 4) Glcβ1- -) receptors neutralize more than 98% of the cytotoxicity of each of the Stx types associated with human disease.

In some embodiments, chimeric LPS is incorporated into the outer membrane of the sentinels. In certain embodiments, the (ma) gene of LPS is mutated, causing the LPS core to be truncated and to terminate in Glc. In certain embodiments, two Neisseria galactosyl-transferase genes (lgtC and lgtE) are inserted, directing the addition of two Gal residues to the Glc acceptor, generating a chimeric LPS terminating in Gal(α1, 4)Gal(β1, 4)Glc, which is the Stx receptor. This in turn prevents Stx from binding similar glycolipid receptors on the surface of neighboring cells such as enterocytes.

As the second aspect of the two-pronged approach, sentinel/killer cells can express Shigella lambdoid phage specific receptor, YaeT, (Smith et al., (2007) J Bacteriology 189:7223; Schmidt, (2001), Research in microbiology, 152:687-695) to absorb phage containing the virulence genes and Stx genes. In some embodiments, incoming phage, along with AI-3, provides the sentinels sufficient proof of Shigella existence. In some embodiments, the sentinel/killer cells sense the lytic phase of the incoming phage by having the same phage promoter P_{L}, activated by N protein, control phage and pathogen killing. In some embodiments, a positive feedback regulator is added on P_{L} after phage detection to maintain FlgM-Bacteriocin synthesis until the pathogen is effectively destroyed.

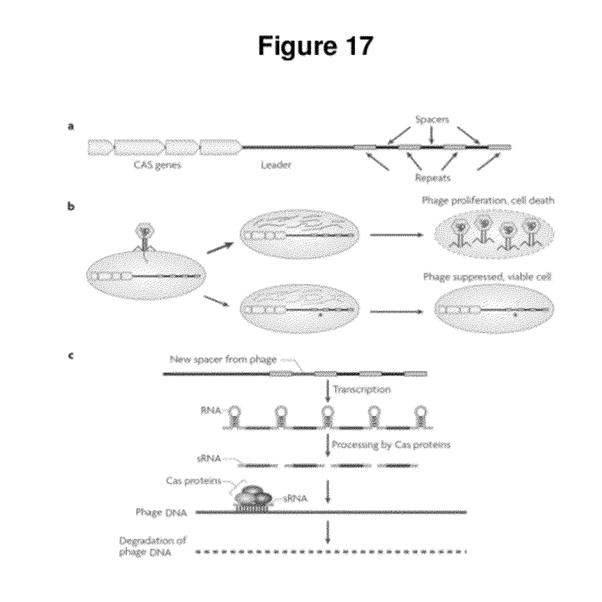

In some embodiments, sentinel/killer cells are engineered to immediately express Clustered Regularly Interspaced Short Palindromic Repeats (CRISPRs) sequences (Sorek et al., (2008) Nature Reviews Microbiology 6:181-186; Pul et al., (2010) Molecular Microbiology, 9999; Marraffini et al., (2010) Nature 463:568-571; Marraffini et al., (2010) Nature Reviews Genetics 11:181-190; Labrie et al., (2010) Nature Reviews Microbiology; Barrangou et al. (2007) Science 315:1709) specific to incoming phage DNA once the phage enters the lytic phase. As used herein, a CRISP is a small repeated sequence separated by short spacer sequences that matches bacteriophage and specifies the targets of interference, a mechanism similar but not homologous to RNAi in eukarayotes (FIG. 17). Engineered CRISPRs have been shown to confer phage resistance (Marraffini et al., (2010) Nature 463:568-571; Marraffini et al., (2010) Nature Reviews Genetics 11:181-190; Labrie et al., (2010) Nature Reviews Microbiology). The repeat-spacer array is transcribed into a long RNA, and the repeats assume a secondary structure.

Without wishing to be bound by any theory, in some embodiments, Cas (CRISPR-associated) proteins naturally present in the sentinel/killer cells recognize the sequence or structure of the repeats and process the RNA to produce small RNAs (sRNAs), each of which contains a spacer and two half repeats. The sRNAs, complexed with additional Cas proteins, base-pair with phage nucleic acids, leading to their degradation.

In some embodiments, CRISPR is engineered to target genes of the phage, such as lytic gene lys, Shiga toxin gene Stx, and/or replication and proliferation genes o and p. In certain embodiments, having P_{L} on a high copy number plasmid further helps titrate away N and prevents expression of phage genes before CRISPR.

In certain embodiments, the sentinel/killer cells are also engineered to express Shigella specific bacteriocin on a plasmid, such as a high copy number plasmid.

One of the advantages of the sense-and-destroy system over previously described systems is the ability to kill a pathogen safely. For example, in some embodiments of the sense-and-destroy system described herein, the bacteria are killed without lysis in order to reduce or prevent toxin release and septic shock from the LPS in the outer membrane. In some embodiments, this is achieved by coupling secretion of engineered Shigella specific colicin (such as colicin U (Smajs et al., (1997) J Bacteriology 179:4919; Cascales et al., (2007) Microbiology and Molecular Biology Reviews 71:158) with CRISPR expression. Without wishing to be bound by any theory, once the colicins are released into the extracellular space, the Receptor Domain of the bacteriocin binds a specific receptor on the outer membrane of the target cell. Then the Translocase Domain forms a complex with the tol receptors on the surface of the cell and facilitates release of the Immunity Protein bound to the Killing/Nuclease Domain. The Killing Domain then enters the target cell and degrades the DNA/RNA without disrupting the outer membrane. Hence, this Shigella antimicrobial approach reduces the possibility of septic shock.

In some embodiments, the Receptor and Translocase Domains of colicin U are fused to the nuclease and immunity domains of colicin E3 produced by E. coli. This allows the new hybrid colicin, named herein “CoShi,” to recognize and specifically kill Shigella strains while leaving the producing strain unharmed.

Aspects of the invention also encompass other types of secreted factors that target and destroy specific pathogens. In some embodiments, the secreted factor is an antimicrobial peptide. Several non-limiting examples of antimicrobial peptides include magainins, alamethicin, pexiganan, polyphemusin, human antimicrobial peptide, LL-37, defenses, protegrins or MSI peptides such as MSI-78, MSI-843 or MSI-594.

Aspects of the invention also encompass embodiments wherein a sense-and-destroy cell secretes a factor by cell suicide. In some embodiments, the secreted factor is a chemokine-derived antimicrobial peptide (CDAP).

In some embodiments, the response of a sense-and-destroy cell to the presence of a pathogen is to kill the pathogen. However, in other embodiments, the response that is generated is not to kill the pathogen. The response can be any kind of therapeutic activity. For example, in some embodiments, the response is to sequester a toxin. In some embodiments, the response is to secrete a factor such as a protein or small molecule.

Aspects of the invention relate to recombinant cells that can sense and destroy specific pathogens. It should be appreciated that the invention encompasses any type of recombinant cell, including prokaryotic and eukaryotic cells. In some embodiments the recombinant cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In certain embodiments, the E. coli cell is an E. coli Nissle 1917 (EcN) cell.

In other embodiments, the recombinant cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. In certain embodiments, the yeast strain is a S. cerevisiae strain. Other non-limiting examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

The recombinant cell can also be an algal cell, a plant cell, an insect cell or an animal cell. In certain embodiments the cell is a mammalian cell such as a rodent cell or a human cell. The cell can be a cell that is associated with the immune system such as a neutrophil, eosinophil, basophil, lymphocyte, monocyte, macrophage or dendritic cell. In some embodiments, the cell is a lymphocyte, such as a B cell, a T cell or a natural killer cell. In certain embodiments, the T cell is a T helper cell (Th cell), a Cytotoxic T cell (CTL or CD8+ T cell), a Memory T cell, a 78 T cell (gamma delta T cell), a Natural killer T cell (NKT cell) or a Regulatory T cell (also called Suppressor T cell). In other embodiments, the cell is a mast cell, a Langerhans cell, a fibroblast, an epithelial cell or a mesothelial cell.

The recombinant cells described herein that sense and destroy specific pathogens are also referred to herein as sentinel cells and/or killer cells. As used herein, a sentinel or killer cell is a cell that can sense and destroy at least one specific pathogen. Cells associated with the invention, and methods of using such cells, are also referred to herein as “sense-and-destroy systems.”

Aspects of the invention relate to the destruction of pathogens. As used herein, a pathogen refers to a biological agent that causes infection and/or disease in its host. Any type of pathogen is compatible with aspects of the invention. In some embodiments, the pathogen is a bacterial pathogen, a viral pathogen, a fungal pathogen, a protist, a parasite or a prion.

Several non-limiting examples of bacterial pathogens include bacteria belonging to the following genera: Bordetella (including, for example, Bordetella pertussis), Borrelia (including, for example, Borrelia burgdorferi), Brucella (including, for example, Brucella abortus, Brucella canis, Brucella melitensis and Brucella suis), Campylobacter (including, for example Campylobacter jejuni), Chlamydia and Chlamydophila (including, for example, Chlamydia pneumoniae, Chlamydia trachomatis, and Chlamydophila psittaci), Clostridium (including, for example, Clostridium botulinum, Clostridium difficile, Clostridium perfringens and Clostridium tetani), Corynebacterium (including, for example, Corynebacterium diphtheriae), Enterococcus (including, for example, Enterococcus faecalis and Enterococcus faecium), Escherichia (including, for example, Escherichia coli), Francisella (including, for example, Francisella tularensis), Haemophilus (including, for example, Haemophilus influenzae), Helicobacter (including, for example, Helicobacter pylori), Legionella (including, for example, Legionella pneumophila), Leptospira (including, for example, Leptospira interrogans), Listeria (including, for example Listeria monocytogenes), Mycobacterium (including, for example, Mycobacterium leprae and Mycobacterium tuberculosis), Mycoplasma (including, for example, Mycoplasma pneumoniae), Neisseria (including, for example, Neisseria gonorrhoeae and Neisseria meningitis), Pseudomonas (including, for example, Pseudomonas aeruginosa), Rickettsia (including, for example, Rickettsia rickettsii), Salmonella (including, for example, Salmonella typhi and Salmonella typhimurium), Shigella (including, for example, Shigella sonnei), Staphylococcus (including, for example, Staphylococcus aureus, Staphylococcus epidermis and Staphylococcus saprophyticus), Streptococcus (including, for example, Streptococcus agalactiae, Streptococcus pneumoniae and Streptococcus pyogenes), Treponema (including, for example Treponema pallidum), Vibrio (including, for example Vibrio cholerae) and Yersinia (including, for example, Yersinia pestis). In some embodiments, the bacterial pathogen is an antibiotic-resistant bacterial pathogen.

Several non-limiting examples of diseases caused by bacterial pathogens include tuberculosis, cholera, dysentery, pneumonia, tetanus, typhoid fever, diptheria, syphilis, congential syphilis, leprosy, bacterial meningitis, sepsis, anthrax, whooping cough, lyme disease, brucellosis, acute enteritis, community-acquired respiratory infection, nongonococcal urethritis, lymphogranuloma venereum, trachoma, inclusion conjunctivitis of the newborn, psittacosis, botulism, pseudomembranous colitis, gas gangrene, food poisoning, anaerobic cellulites, nosocomial infections, urinary tract infections, diarrhea, hemorrhagic colitis, hemolytic-uremic syndrome, tularemia, upper respiratory tract infections, bronchitis, peptic ulcers, legionnaire's disease, pontiac fever, leptospirosis, listeriosis, tuberculosis, gonorrhea, ophthalmia neonatorum, septic arthritis, meningococcal disease, Waterhouse-Friderichsen syndrome, Pseudomonas infection, rocky mountain spotted fever, typhoid fever type salmonellosis (dysentery, colitis), Salmonellosis with gastroenteritis and/or enterocolitis, bacillary dysentery/Shigellosis, coagulase-positive staphylococcal infections (such as impetigo, acute infective endocarditis, septicemia, necrotizing pneumonia, and toxinoses such as toxic shock syndrome or Staphylococcal food poisoning), cystitis, septicemia, endometritis, otitis media, sinusitis, Streptococcal pharyngitis, scarlet fever, rheumatic fever, erysipelas, puerperal fever, necrotizing fascilitis, bubonic plague and pneumonic plague.

Several non-limiting examples of viral pathogens include viruses belonging to the following families: Adenoviridae (including, for example, adenovirus), Picornaviridae (including, for example, coxsackievirus, hepatitis A virus, poliovirus and rhinovirus), Herpesviridae (including, for example, herpes simplex type 1, herpes simplex type 2, Varicella-zoster virus, Epstein-barr virus, human cytomegalovirus, and human herpesvirus type 8), Hepadnaviridae (including, for example, hepatitis B virus), Flaviviridae (including, for example, hepatitis C virus, yellow fever virus, dengue virus, and West Nile virus), Retroviridae (including, for example, human immunodeficiency virus (HIV)), Orthomyxoviridae (including, for example, influenza virus), Paramyxoviridae (including, for example, measles virus, mumps virus, parainfluenza virus, respiratory syncytial virus and human metapneumovirus), Papillomaviridae (including, for example, papillomavirus), Rhabdoviridae (including, for example, rabies virus), Togaviridae (including, rubella virus) and Parvoviridae (including, for example, human bocavirus and parvovirus B19).

Several non-limiting examples of diseases caused by viral pathogens include: acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection, gingivostomatitis, tonsillitis, pharyngitis, primary HSV-2 infection, latent HSV-2 infection, aseptic meningitis, infectious mononucleosis, cytomegalic inclusion disease, Kaposi's sarcoma, Castleman disease, primary effusion lymphoma, AIDS, influenza, Reye syndrome, measles, postinfectious encephalomyelitis, mumps, hyperplastic epithelial lesions, laryngeal papillomas, epidermodysplasia verruciformis, croup, pneumonia, bronchiolitis, common cold, rabies, German measles, congenital rubella, varicella and herpes zoster.

Several non-limiting examples of pathogenic fungi include fungi belonging to the genera: Candida (including, for example, Candida albicans), Aspergillus (including, for example, Aspergillus fumigatus, Aspergillus flavus and Aspergillus clavatus), Cryptococcus (including, for example, Cryptococcus neoformans and Cryptococcus gattii), Histoplasma (including, for example, Histoplasma capsulatum), Pneumocystis (including, for example, Pneumocystis jirovecii or Pneumocystis carinii), and Stachybotrys (including, for example, Stachybotrys chartarum).

Non-limiting examples of diseases or disorders caused by fungal pathogens include: respiratory damage, allergic diseases, Aspergillosis, meningitis, meningo-encephalitis, histoplasmosis and pneumonia. Fungal infections are also referred to as mycosis. There are several classifications of mycosis including superficial, cutaneous, subcutaneous, systemic mycoses due to primary pathogens and systemic mycoses due to opportunistic pathogens.

Pathogens can also include protists. For example, protists of the genus Plasmodium cause malaria. Non-limiting examples of species of Plasmodium parasites include Plasmodium falciparum, Plasmodium vivax, Plasmodium ovale, and Plasmodium malariae. Protists of the genus Phytophthora, such as Phytophthora infestans, cause potato blight.

Pathogens can also include parasites, such as parasitic worms or helminthes including the categories of cestodes, nematodes and trematodes. Pathogens can also include prions (proteinaceous infectious particles) which cause diseases such as bovine spongiform encephalopathy and Creutzfeldt-Jakob disease.

It should be appreciated that the genes and proteins, or portions thereof, recombinantly expressed in the cells described herein can be obtained from a variety of sources. As one of ordinary skill in the art would be aware, homologous genes can be obtained from multiple species and can be identified by homology searches, for example through a protein BLAST search, available at the NCBI internet site (www.ncbi.nlm.nih.gov). Additionally, as one of ordinary skill in the art would be aware, any suitable functional screen or assay could be used to identify functional homologs of these genes. Genes recombinantly expressed herein, or portions thereof can be PCR amplified from DNA from any source that contains the given gene or portions thereof. In some embodiments, one or more of the genes or portions thereof is synthetic.

Aspects of the invention include strategies to optimize production of a secreted factor from a cell. Optimized production of a secreted factor refers to producing a higher amount of the secreted factor following pursuit of an optimization strategy than would be achieved in the absence of such a strategy. In embodiments that employ recombinant cells, one strategy for optimization is to increase or decrease expression levels of the recombinant genes through selection of appropriate promoters and/or ribosome binding sites. In some embodiments this may include the selection of high-copy number plasmids, or low or medium-copy number plasmids. The step of transcription termination can also be targeted for regulation of gene expression, through the introduction or elimination of structures such as stem-loops.

In some embodiments it may be advantageous to use a cell that has been optimized for production of one or more secreted factors. For example, it may be optimal to mutate the cell prior to or after introduction of recombinant gene products. In some embodiments, screening for mutations that lead to enhanced production of one or more secreted factors may be conducted through a random mutagenesis screen, or through screening of known mutations. In some embodiments, shotgun cloning of genomic fragments can be used to identify genomic regions that lead to an increase in production of one or more secreted factors, through screening cells or organisms that have these fragments for increased production of one or more secreted factors. In some cases one or more mutations may be combined in the same cell or organism. In some embodiments, one or more flagellar genes in the cell are mutated or deleted. In certain embodiments, the flagellar genes that are mutated or deleted are within the FliCDST operon.

Optimization of protein expression may also require in some embodiments that a gene be modified before being introduced into a cell such as through codon optimization for expression in a bacterial cell. Codon usages for a variety of organisms can be accessed in the Codon Usage Database (http://www.kazusa.or.jp/codon/).

Protein engineering can also be used to optimize expression or activity of a protein. In certain embodiments, a protein engineering approach can include determining the three dimensional (3D) structure of a protein such as an enzyme or constructing a 3D homology model for the protein based on the structure of a related protein. Based on 3D models, mutations in a protein can be constructed and incorporated into a cell or organism, which can then be screened for an increased production of one or more secreted proteins. In some embodiments, production of a secreted protein in a cell can be increased through manipulation of proteins that act in the same pathway as the proteins associated with the invention. For example in some embodiments, it may be advantageous to increase expression of a protein or other factor that acts upstream of a target protein such as a protein encoded for by one of the genes that is recombinantly expressed in cells associated with the invention. This can be achieved by over-expressing the upstream factor using any standard method.

Aspects of the invention thus involve recombinant expression of genes discussed above, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C. There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general, homologs and alleles typically will share at least 75% nucleotide identity to the sequences of nucleic acids. For example, in some embodiments, homologs and alleles will share at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more than 99% nucleotide identity. In general, homologs and alleles typically will share at least 80% amino acid identity to the sequences of polypeptides. For example, in some embodiments, homologs and alleles will share at least 80%, 81%, 82%, 83%, 84%, 85%, 86%, 87%, 88%, 89%, 90%, 91%, 92%, 93%, 94%, 95%, 96%, 97%, 98%, 99% or more than 99% amino acid identity.

The homology can be calculated using various, publicly available software tools developed by NCBI (Bethesda, Md.) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also encompasses isolated polypeptides such as novel chimeric bacteriocin proteins. As used herein, the terms “protein” and “polypeptide” are used interchangeably and thus the term polypeptide may be used to refer to a full-length polypeptide and may also be used to refer to a fragment of a full-length polypeptide. As used herein with respect to polypeptides, proteins, or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure.

The term “substantially pure” means that the proteins or polypeptides are essentially free of other substances with which they may be found in production, nature, or in vivo systems to an extent practical and appropriate for their intended use. Substantially pure polypeptides may be obtained naturally or produced using methods described herein and may be purified with techniques well known in the art. Because an isolated protein may be admixed with other components in a preparation, the protein may comprise only a small percentage by weight of the preparation. The protein is nonetheless isolated in that it has been separated from the substances with which it may be associated in living systems, i.e. isolated from other proteins.

The invention also encompasses nucleic acids that encode for any of the polypeptides described herein, libraries that contain any of the nucleic acids and/or polypeptides described herein, and compositions that contain any of the nucleic acids and/or polypeptides described herein. It should be appreciated that libraries containing nucleic acids or proteins can be generated using methods known in the art. A library containing nucleic acids can contain fragments of genes and/or full-length genes and can contain wild-type sequences and mutated sequences. A library containing proteins can contain fragments of proteins and/or full length proteins and can contain wild-type sequences and mutated sequences. It should be appreciated that the invention encompasses codon-optimized forms of any of the nucleic acid and protein sequences described herein.

The invention embraces variants of polypeptides. As used herein, a “variant” of a polypeptide is a polypeptide which contains one or more modifications to the primary amino acid sequence of the polypeptide. Modifications which create a variant can be made to a polypeptide 1) to reduce or eliminate an activity of a polypeptide; 2) to enhance a property of a polypeptide; 3) to provide a novel activity or property to a polypeptide, such as addition of an antigenic epitope or addition of a detectable moiety; or 4) to provide equivalent or better binding between molecules (e.g., an enzymatic substrate). Modifications to a polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the amino acid sequence. One of skill in the art will be familiar with methods for predicting the effect on protein conformation of a change in protein sequence, and can thus “design” a variant of a polypeptide according to known methods. One example of such a method is described by Dahiyat and Mayo in Science 278:82-87, 1997, whereby proteins can be designed de novo. The method can be applied to a known protein to vary only a portion of the polypeptide sequence. By applying the computational methods of Dahiyat and Mayo, specific variants of a polypeptide can be proposed and tested to determine whether the variant retains a desired conformation. In general, variants include polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a polypeptide by eliminating proteolysis by proteases in an expression system (e.g., dibasic amino acid residues in yeast expression systems in which KEX2 protease activity is present).

Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of variant polypeptides can be tested by cloning the gene encoding the variant polypeptide into a bacterial or mammalian expression vector, introducing the vector into an appropriate host cell, expressing the variant polypeptide, and testing for a functional capability of the polypeptides as disclosed herein.

The skilled artisan will also realize that conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In general, it is preferred that fewer than all of the amino acids are changed when preparing variant polypeptides. Where particular amino acid residues are known to confer function, such amino acids will not be replaced, or alternatively, will be replaced by conservative amino acid substitutions. In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. It is generally preferred that the fewest number of substitutions is made. Thus, one method for generating variant polypeptides is to substitute all other amino acids for a particular single amino acid, then assay activity of the variant, then repeat the process with one or more of the polypeptides having the best activity.

Conservative amino-acid substitutions in the amino acid sequence of a polypeptide to produce functionally equivalent variants of the polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

A polypeptide or fragment thereof described herein can be synthetic. As used herein, the term “synthetic” means artificially prepared. A synthetic polypeptide is a polypeptide that is synthesized and is not a naturally produced polypeptide molecule (e.g., not produced in an animal or organism). It will be understood that the sequence of a natural polypeptide (e.g., an endogenous polypeptide) may be identical to the sequence of a synthetic polypeptide, but the latter will have been prepared using at least one synthetic step.

In some embodiments, one or more of the genes associated with the invention is expressed in a recombinant expression vector. As used herein, a “vector” may be any of a number of nucleic acids into which a desired sequence or sequences may be inserted, such as by restriction and ligation, for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that comprises any of the genes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in some embodiments include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

One or more nucleic acid molecules that encode genes associated with the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing one or more nucleic acid molecules associated with the claimed invention also may be accomplished by integrating the one or more nucleic acid molecules into the genome.

Aspects of the invention relate to the destruction of pathogens. In some embodiments, the pathogen is in vivo. It should be appreciated that an in vivo pathogen compatible with aspects if the invention can be anywhere inside the body. In some embodiments, the pathogen is within the gastrointestinal tract. In some embodiments, the pathogen is within the lungs. In some embodiments, the pathogen is present at a low cell density while in other embodiments, the pathogen is present at a high cell density. In some embodiments, the pathogen is outside the body. For example in some embodiments, the pathogen is on equipment such as medical equipment. Methods described herein can be used for sterilization such as the sterilization of medical equipment. In some embodiments, a cell described herein is a component of a biospray. As used herein, a biospray refers to a composition that can be sprayed onto surfaces or devices such as food contact surfaces or medical equipment for the purposes of cleansing, sanitizing and/or disinfecting. A biospray can be used alone or in combination with other agents. In some embodiments, a cell described herein is a component of an artificial immune system.

Methods described herein can also be applied for treatment and/or prevention of conditions or diseases. As used herein, the terms “treat,” “treated,” or “treating” when used with respect to a disease, such as an infectious disease, refers to a prophylactic treatment that increases the resistance of a subject to development of the disease or, in other words, decreases the likelihood that the subject will develop the disease as well as a treatment after the subject has developed the disease in order to fight the disease or prevent the disease from becoming worse. Treatment after a condition (e.g., an infectious disease) has started aims to reduce, ameliorate or altogether eliminate the condition, and/or its associated symptoms, or prevent it from becoming worse. Treatment of subjects before a condition (e.g., an infectious disease) has started (i.e., prophylactic treatment) aims to reduce the risk of developing the condition and/or lessen its severity if the condition does develop. In some embodiments, treatment of a subject who has a disease can lead to partial or complete curing of the subject of the disease. As used herein, the terms “cure” or “curing” refers to reducing or eliminating the symptoms of a disease in a subject. In some embodiments, prophylactic treatment of a subject can lead to prevention of a disease in a subject.

As used herein, the term “prevent” refers to the prophylactic treatment of a subject who is at risk of developing a condition (e.g., an infectious disease) resulting in a decrease in the probability that the subject will develop the disorder, and/or to the inhibition of further development of an already established disorder. As used herein, the term “protect against” refers to the prophylactic treatment of any subject at any time with the object of preventing the possibility of the development of a disorder such as an infectious disease, resulting in a decrease in the probability that the subject will develop the disorder. In some embodiments, prevention of a disease in a subject or protection against a disease in a subject will result in the subjected developing reduced or no symptoms of the disease relative to control subjects in which the disease has not been prevented or protected against.

The term “effective amount” of a cellular sense-and-destroy system described herein refers to the amount necessary or sufficient to realize a desired biologic effect. For example, an effective amount for treating an infectious disease is that amount sufficient to prevent an increase in symptoms of the infectious disease or that amount necessary to decrease the amount of further damage that would otherwise occur in the absence of the cellular sense-and-destroy system. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular composition being administered, the size of the subject, or the severity of the disease or condition. One of ordinary skill in the art can empirically determine the effective amount of a particular composition of the invention without necessitating undue experimentation.

Compositions associated with the invention can be delivered to the subject on an as needed or desired basis. For instance a subject may self administer compositions as desired in order to protect against or treat or prevent a condition or disease such as an infectious disease. Additionally, a physician or other health care worker may select a delivery schedule. In other embodiments of the invention, the compositions are administered on a routine schedule. A “routine schedule” as used herein, refers to a predetermined designated period of time. The routine schedule may encompass periods of time which are identical or which differ in length, as long as the schedule is predetermined. For instance, the routine schedule may involve administration of the composition on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the composition on a daily basis for the first week, followed by a monthly basis for several months, and then every three months after that. Any particular combination would be covered by the routine schedule as long as it is determined ahead of time that the appropriate schedule involves administration on a certain day.

In some embodiments, compositions described herein are components of a water supply system or components of a food product such as a probiotic or nutraceutical. In such embodiments, the compositions may be ingested by subjects at any time and not necessarily as part of a structured administration regimen.

Compositions comprising cells associated with the invention may be administered alone or in any appropriate pharmaceutical carrier, such as a liquid, for example saline, or a powder, for administration in vivo. The compositions may be formulated. The formulations of the invention can be administered in pharmaceutically acceptable solutions, which may routinely contain, for example, pharmaceutically acceptable concentrations of salt, buffering agents, preservatives, compatible carriers, adjuvants, and optionally other therapeutic ingredients.

For use in therapy, an effective amount of the compositions can be administered to a subject by any mode that delivers the cells to the desired region of the body. Administering the compositions, such as within a pharmaceutical composition, may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

It is well known to those skilled in the art that cellular systems associated with the invention may be administered to patients using a full range of routes of administration. As an example, compositions comprising such cells may be blended with direct compression or wet compression tableting excipients using standard formulation methods. The resulting granulated masses may then be compressed in molds or dies to form tablets and subsequently administered via the oral route of administration. Alternately particle granulates may be extruded, spheronized and administered orally as the contents of capsules and caplets. Tablets, capsules and caplets may be film coated to alter dissolution of the delivery system (enteric coating) or target delivery to different regions of the gastrointestinal tract. Additionally, cells described herein may be orally administered as suspensions in aqueous fluids or sugar solutions (syrups) or hydroalcoholic solutions (elixirs) or oils. The particles may also be administered directly by the oral route without any further processing.

The cells of the invention may be systemically administered in combination with a pharmaceutically acceptable vehicle such as an inert diluent or an assimilable edible carrier. They may be enclosed in hard or soft shell gelatin capsules or compressed into tablets. For oral therapeutic administration, the active compound may be combined with one or more excipients and used in the form of ingestible tablets, buccal tablets, troches, capsules, elixirs, suspensions, syrups, wafers, and the like. In some embodiments, such compositions and preparations should contain at least 0.1% of active compound, such as calcium. The percentage of the compositions and preparations may, of course, be varied and may in some embodiments be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

To ensure full gastric resistance a coating impermeable to at least pH 5.0 is helpful. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

Compositions comprising cells associated with the invention may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In some embodiments the compositions of the invention are not encapsulated or formulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration, the cells of the invention will generally be administered as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The compositions of the inventions may include a physiologically or pharmaceutically acceptable carrier, excipient, or stabilizer mixed with the particles. The term “pharmaceutically acceptable” means a non-toxic material that does not interfere with the effectiveness of the biological activity of the active ingredients. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being co-mingled with the compounds of the present invention, and with each other, in a manner such that there is no interaction which would substantially impair the desired pharmaceutical efficiency. A pharmaceutical preparation is a composition suitable for administration to a subject. Such preparations are usually sterile and prepared according to GMP standards, particularly if they are to be used in human subjects. In general, a pharmaceutical composition or preparation comprises the cells, and optionally agents of the invention and a pharmaceutically-acceptable carrier, wherein the agents are generally provided in effective amounts.

Cells may also be suspended in non-viscous fluids and nebulized or atomized for administration of the dosage form to nasal membranes. Cells may also be delivered parenterally by either intravenous, subcutaneous, intramuscular, intrathecal, intravitreal or intradermal routes as sterile suspensions in isotonic fluids.

Cells may also be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the nanoparticle or microparticle (see, for example, Sciarra and Cutie, “Aerosols,” in Remington's Pharmaceutical Sciences, 18th edition, 1990, pp. 1694-1712; incorporated by reference).

Some specific examples of commercially available devices suitable for such means of administration include the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

Composition comprising cells associated with the invention, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Composition comprising cells associated with the invention can be used as stand alone therapies. A stand alone therapy is a therapy in which a prophylactically or therapeutically beneficial result can be achieved from the administration of a single agent or composition. Accordingly, compositions disclosed herein can be used alone in the prevention or treatment of infectious disease, because the compositions are capable of detecting and destroying pathogens responsible for the development of infectious disease. Some of the methods described herein relate to the use of such compositions as a stand alone therapy, while others related to the use of such compositions in combination with other therapeutic agents. Compositions described herein can also be components of vaccines.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### Sense and Destroy for P. Aeruginosa (PAO-1)

In 1917, a German professor named Alfred Nissle isolated a strain of E. coli from the feces of a World War I soldier who did not develop enterocolitis during a severe outbreak of shigellosis. Since antibiotics had not yet been discovered, Nissle used the strain with considerable success in acute cases of infectious intestinal diseases (such as salmonellosis and shigellosis). E. coli Nissle 1917 (EcN) is still used today and is one of the few examples of a non-LAB probiotic. This strain is particularly helpful in the management of gastrointestinal infectious disorders and infections affecting the urinary tract. Since then, the genome of EcN has been fully sequenced and it has exhibited a number of fitness factors, including for example, microcins, adhesins, and proteases. Besides these, it contains at least 6 different iron-uptake systems (enterobactin, salmochelin, aerobactin, yersiniabactin, EfeU) and lacks prominent virulence factors (e.g., hemolysin, P-fimbrial adhesions etc.). The strong antagonism of EcN toward other members of the intestinal microbiota is based in part on the production of microcins, later identified as microcins H47 and M[1]. This remarkable combination of fitness factors and lack of virulence factors coupled with the demonstration of probiotic properties in several animal models for experimental colitis makes this strain a promising choice for sentinel chassis.

### Detection of Pathogenic Bacteria

In the canonical Gram-negative Quorum Sensing (QS) system, an I-protein synthase produces acylated homoserine lactone (AHL) autoinducers which diffuse freely between the cytoplasm and the environment, then directly interact with R-protein transcriptional regulators to control the expression of target genes. The design for sentinel/killer cells includes an R-protein (regulated by an inducible promoter) which binds AHL produced from the target pathogen and promotes expression of E/GFP and lysin.

To detect P. aeruginosa, synthetic gene networks were constructed in E. coli that express GFP in response to 3OC₁₂HSL, the autoinducer produced by P. aeruginosa QS synthase LasI (FIG. 2A). Receiving cells were grown to an OD of 0.5 and then induced and incubated with different concentrations of 3OC₁₂HSL for 3 hours. Fluorescence was then measured by flow cytometry. The resulting 3OC₁₂HSL dosage response curve is shown in FIG. 2B.

Once receiving cells that respond well to exogenous 3OC₁₂HSL were engineered, the next question was to determine how well they would respond to AHL directly produced by PAO-1 cells. P. aeruginosa cells were grown to different ODs and the supernatant was filter-sterilized. The concentration of 3OC₁₂HSL in the supernatant is directly proportional to pathogen density. The supernatant contains signals that the pathogen uses for quorum sensing but not the cells themselves. The 3OC₁₂HSL-responsive receiver cells were grown in the supernatant and fluorescence was measured. The graph in FIG. 2C demonstrates the increase in fluorescence output in the receiver cells as a function of pathogen density.

### Secretion of Killer Cells

The suitability of a polypeptide secretion mechanism for killer proteins was investigated. As shown in FIG. 3, in S. typhimurium, flagellar production is controlled by master operon flhDC which is expressed from a class I promoter. The FlhD and FlhC proteins from this operon form a heteromultimeric complex (FlhD4C2) which in turn acts as a transcriptional activator for class II flagellar promoters. Completion and assembly of flagellar motor structure, also known as the HBB, requires transcription of genes controlled by σ70-dependent class II promoters. Upon HBB completion, class III promoters are transcribed by σ28 RNA polymerase, which is specific for flagellar class III promoters. Prior to HBB completion, an anti-σ28 factor FlgM inhibits transcription by σ28 RNA polymerase. Upon HBB completion, FlgM is secreted from the cell through the completed HBB structure and σ28 initiates transcription of genes which polymerize to form flagella on HBB. Hence, flagellin filament genes are only transcribed when there is a functional motor onto which they can be assembled. E. coli has similar flagellar control. This system presents a potential mechanism to secrete the killer protein.

Herein, the killer protein CoPy was fused to FlgM and the fusion protein was placed under the transcriptional control of the las promoter which is only expressed when pathogen is detected in the medium. Secretion of FlgM-CoPy was successfully demonstrated.

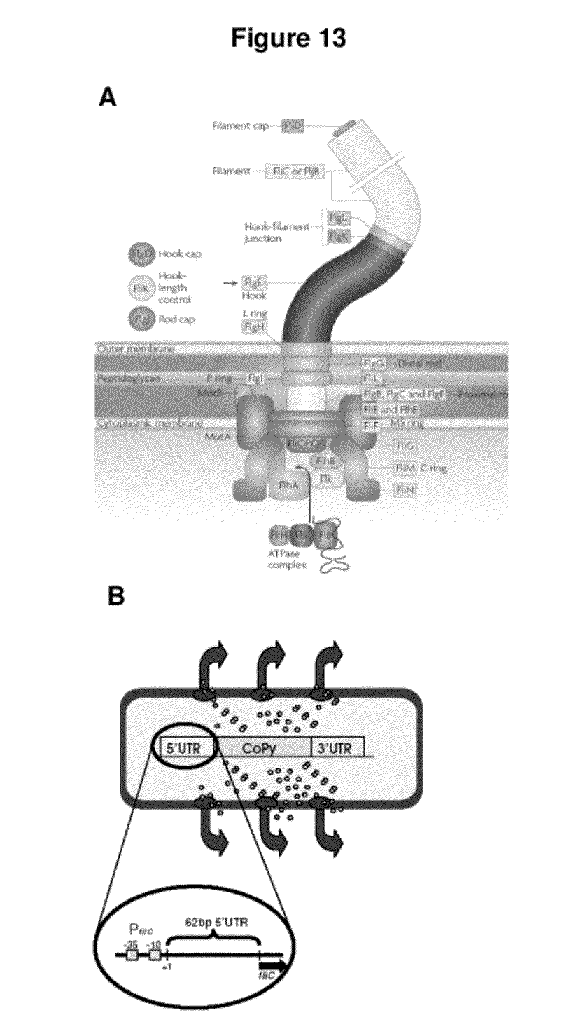

FIG. 13 summarizes FliC based secretion. FIG. 13A depicts a schematic of flagellar proteins. FIG. 13B depicts a schematic of engineering flagellar gene expression (from Chevance et al., Nature Reviews, 2008).

### Selective Destruction of Pathogenic Bacteria Upon Detection

Bacteriocins are highly specific and potent toxins produced by a small portion of the cell population during stressful conditions such as nutrient depletion, overcrowding, stationary phase of growth or high temperatures. Expression and secretion of bacteriocins usually results in rapid elimination of neighboring cells, sometimes of the same species, that are not immune to their effect. Bacteriocins have evolved to parasite various cell surface receptors normally involved in uptake and passage of small nutrient molecules (such as Vitamin B12 and iron) across the outer membrane (OM). BtuB is such a receptor protein in E. coli and Fpy in PAO-1. Colicins (E1) and pyocins (S2), which are naturally expressed lysins in Gram-negative bacteria, target these cell surface receptors. Colicins and pyocins usually have three distinct domains with different functionality. The Recognition Domain binds specific receptors on the surface of the target species. The Translocase Domain translocates the Nuclease Domain into the cell. This nuclease domain can be DNase or RNase which kills a target cell by cleaving its DNA or RNA.

The producing cell is protected from the killing activity of its own colicin because the colicin is produced along with an immunity protein to which it is translationally coupled to the colicin and which forms a tight complex with the nuclease domain. In a target cell, the complex dissociates when the receptor domain binds the corresponding receptor on the target cell.

When the nuclease domain enters the producing cell, it is immediately bound by an immunity protein present in the cytoplasm, preventing nuclease activity. This protection provides the producing cell with an advantage over the target cells which do not express the immunity protein. FIG. 4 shows a schematic representation of the natural colicin killing mechanism. Pyocins are P. aeruginosa bacteriocins and have the same general structure as colicins.

A novel bacteriocin was produced that specifically kills the pathogen PAO-1. To engineer a protein which selectively kills PAO-1, the colicin recognition and translocation domains were replaced with those of pyocin, as demonstrated in FIG. 5. The modified protein, called CoPy, kills only the P. aeruginosa pathogen while the E. coli sentinel is unaffected by the protein since the immunity protein is produced by the sentinel and also due to different cell surface receptors (FIG. 4).

Assays were conducted for toxic effects of CoPy on sentinel cells when it was produced but not secreted. CoPy was cloned under the control of promoter P_{L}tetO₁, a promoter that is regulated by TetR and anhydrotetracycline (aTc). Sentinel cells were grown and CoPy expression was induced with 100 ng/ml aTc. The behavior of these cells was compared to that of cells without CoPy expression (containing pProtet plasmid). Cell density was monitored for approximately 20 hours and it was observed that when CoPy was produced inside E. coli sentinels, it did not kill them.

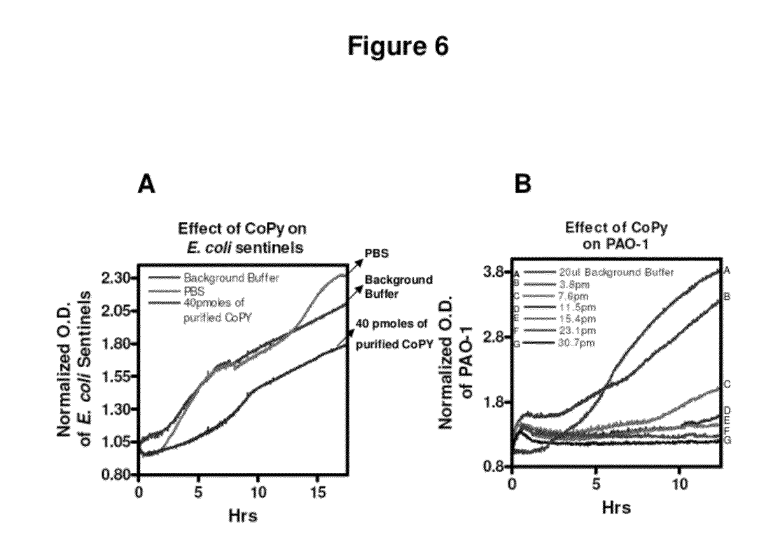

A synthetic gene network was constructed where CoPy was placed under control of the las promoter and LasR was expressed constitutively. To test CoPy killing and specificity, the protein was purified using a HisTag fused to the N-terminus of CoPy. 5 ml of producing cells (DH5alpha-pro with the circuit) were grown to an OD of 0.5, and then CoPy expression was induced using 10 μM 3OC₁₂HSL for 4 hours. Cells were then concentrated and Qiagen Nickel columns were used to purify the protein from the cell pellet. Subsequently, sentinel cells and PAO1 were incubated with various concentrations of purified CoPy for approximately 15 hours. As a control, cells were also incubated with equal amounts of PBS and background buffer used for purifying CoPy. FIGS. 6A and 6B show that CoPy completely halted PAO1 growth while sentinel growth was unaffected by a higher concentration of CoPy.

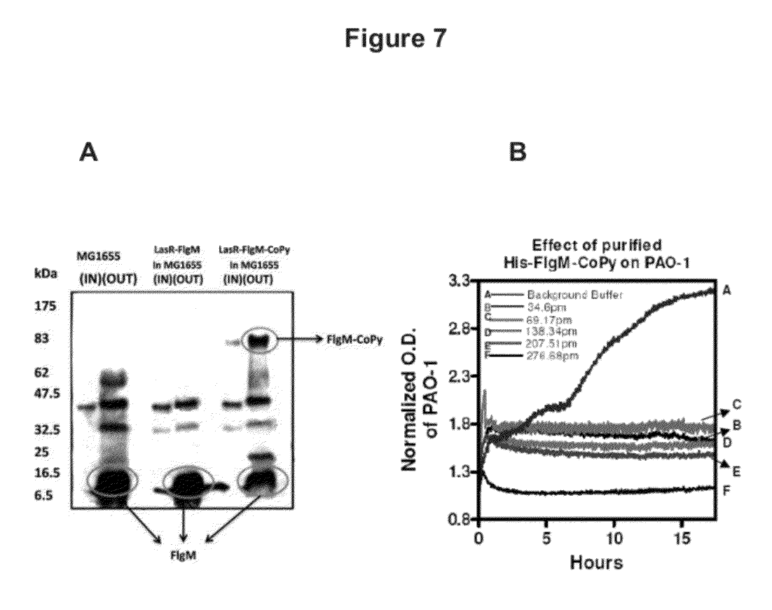

Once the individual sensing and killing components were proven to work, they were combined to test the complete system. FlgM was fused to CoPy and the fusion protein was placed under the transcriptional control of the las promoter which is transcribed only when the pathogen is present in the medium. FIG. 7A shows a Western Blot of FlgM-CoPy that was secreted into the medium. This result demonstrates that the engineered sentinel/killer cells are able to secrete CoPy with the aid of FlgM when sensing 3OC₁₂HSL. To assay sense-and-destroy function, the sentinels were grown to an OD of 0.1 in LB medium and then expression was induced with 10 μM 3OC₁₂HSL. This directs the sentinels to produce and secrete His-FlgM-CoPy. The supernatant was filter-sterilized and the secreted protein was His-purified using nickel columns. Protein concentration was measured using a Bradford Assay. Differing concentrations of the secreted CoPy were used to characterize the dosage response.

Next, a lab strain of E. coli (MG1655) was engineered to secrete the chimeric killer protein, CoPy, in response to 3OC₁₂HSL. To assay whether secreted FlgM-CoPy killed PAO-1, 70 ml of sentinels were grown to an OD of 0.1 and CoPy expression was induced with 10 uM 3OC₁₂HSL for 4 hours. The supernatant was then filter sterilized and secreted His-tagged FlgM-CoPy was purified. The effect of 100 ul of the secreted killer protein was observed on the growth of 100 ul of 0.01 OD PAO-1 using a plate reader for 15 hours (FIG. 8A). The sentinel/killer gene circuit was also integrated into EcN, and killing was compared with FlgM-CoPy from MG1655 and EcN versus PAO-1 grown in background buffer. FlgM-CoPy from both MG1655 and EcN successfully inhibited PAO-1 growth (FIG. 8A). Thus, secreted FlgM-CoPy selectively kills PAO-1.

Next, sentinels (EcN) and PAO-1 were co-cultured on an agar plate and growth and fluorescence were observed. Sentinels express GFP in response to 3OC₁₂HSL produced by PAO-1, while PAO-1 cells constitutively express red fluorescent protein. 10 ul droplets of sentinels and PAO-1 were pipetted on a bed of PAO-1 and the composition of the droplet was observed under a microscope after 10 hours of incubation at 37° C. E. coli in the negative control could detect PAO-1 but did not have the ability to kill them. FIG. 8B shows brightfield, green, and red fluorescent images of PAO-1 with control or sentinel/killer cells. The concentration of PAO-1 (indicated by red fluorescence) was significantly lower, essentially undetectable, in the case where sentinel/killers were present. Green fluorescence is also lower in that case because sentinel GFP expression is dependent on the presence of PAO-1, but the brightfield image shows high cell density, indicating that only sentinels were present.

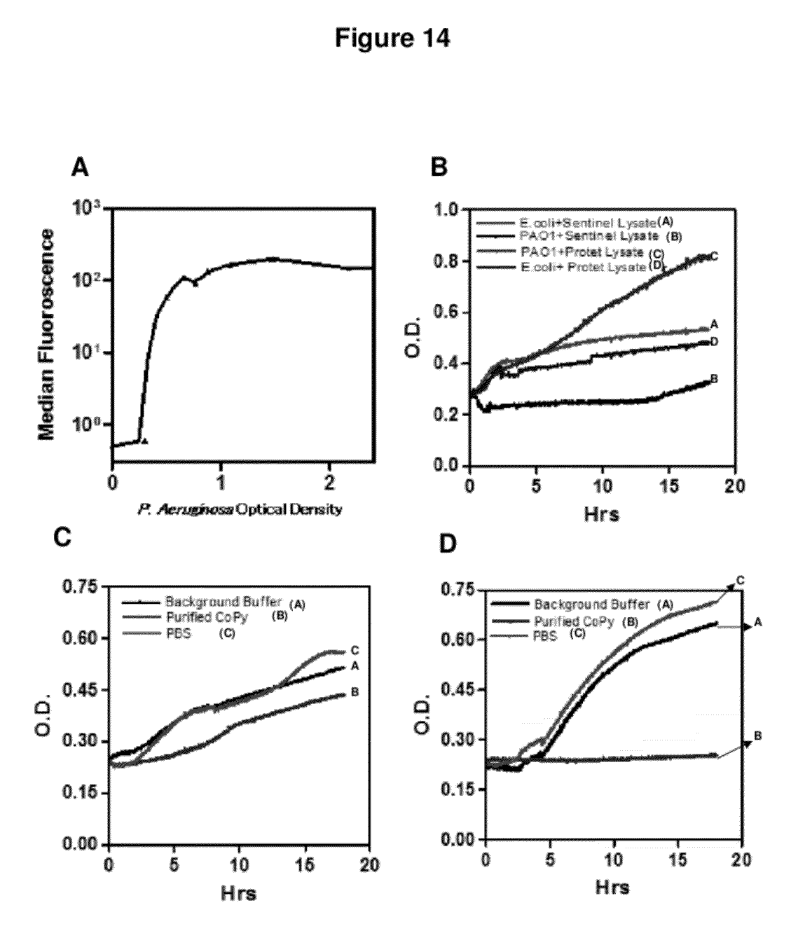

FIG. 14 presents results from plate reader data with E. coli and PAO1. In FIG. 14A, PAO1 were grown to different ODs and then the supernatant was collected and filter-sterilized. Receiver cells were grown in the supernatant for 3 hours and then subjected to FACS. In FIG. 14B, PAO1 and E. coli cells were grown to the same OD separately. Cell lysate was extracted from sentinels (containing CoPy) and from controls (containing pProtet but not CoPy). The ODs of the PAO1 and E. coli cells with lysate was monitored in a plate reader.

In FIG. 14C, E. coli sentinels were grown to an OD of 0.25. CoPy was purified from the sentinels using an N-terminal His-tag. The OD of the E. coli cells was monitored with the purified CoPy, PBS (phosphate buffered saline) and the background buffer used to purify CoPy in a plate reader. In FIG. 14D, PAO1 pathogen was grown to an OD of 0.25. CoPy was purified from the sentinels using an N-terminal fused His-tag. The OD of PAO1 was monitored with the purified CoPy, PBS and the background buffer used to purify CoPy in a plate reader.

### Optimization

Time-dependent dosage response of secreted FlgM-CoPy are evaluated and sentinel concentration required to effectively contain PAO-1 is evaluated. By engineering sentinels to constitutively express GFP, real time monitoring of PAO-1/sentinel co-cultures is performed to characterize their respective ratios. Various aspects of system performance (e.g. secretion and FlgM-CoPy killing efficiencies) are optimized. The toxic effect of P. aeruginosa on mammalian epithelial cell growth in tissue culture as well as other existing cell lines is characterized and the ability of EcN sentinel/killer cells to rescue mammalian cells by killing the pathogen is evaluated. The system is tested in vivo in a mouse model.

Response sensitivity of the las system is improved by coupling it to a signal amplifier genetic circuit. This circuit amplifies the response by inserting cI downstream of a las promoter allowing λ_{P(R)} to regulate killing protein expression. CI is a very efficient transcriptional repressor. Even small increases in CI levels, normally undetectable by fluorescence microscopy, yields large changes in λ_{P(R)} activity and hence easily observable changes in the final signal output. The circuit is fine-tuned by measuring the performance of several different λ_{P(R)} mutants. The mutants are titrated with exogenous 3OC₁₂HSL until highly sensitive detection capabilities are achieved. The limits of P. aeruginosa detection are characterized by co-culturing the pathogen with E. coli harboring the best signal amplifier. This analysis is carried out using a microplate reader with custom dual wells that have a permeable 0.22μ membrane between them. Wildtype P. aeruginosa are grown in one well, while signal amplifying E. coli sentinels are grown in the adjoining well. 3OC₁₂HSL diffuses freely through the connecting permeable membrane. This microplate reader allows for the determination of the minimal P. aeruginosa culture density required for detection by signal amplifying E. coli sentinels.

### Example 2

### V. Cholerae Sense and Destroy

The V. cholerae pathogen uses two QS pathways, one broad and the other species specific. Many pathogens, including V. cholerae, produce and respond to a set of interconverting molecules, together called AI-2, that are derived from the shared precursor 4,5-dihydroxy-2,3-pentanedione (DPD) that is synthesized by the LuxS enzyme. CAI-1, (S)-3-hydroxytridecan-4-one, is the major species specific quorum sensing signal in V. cholera [3,4]. Detection of the V. Cholerae autoinducers occurs through membrane-bound histidine kinases that act as cognate receptors for the two autoinducers, as shown in FIG. 9. AI-2 is detected by the periplasmic protein LuxP in a complex with LuxQ, while CAI-1 is detected by CqsS. LuxQ and CqsS are bi-functional two-component enzymes that possess both kinase and phosphatase activities.

At low cell density (LCD), these two proteins are devoid of their respective ligands and act as kinases, resulting in phosphorylation of histidine residues by ATP. The phosphate group is next transferred to the conserved aspartate residue located in the receiver domain of each receptor. Phosphate from both the receptors is subsequently transduced to a single phosphotransfer protein, LuxU, which transfers the phosphate to a response regulator called LuxO. LuxO belongs to the NtrC family of response regulators and requires phosphorylation to act as a transcriptional activator. Phosphorylated LuxO (LuxO-P) activates transcription of genes encoding four small regulatory RNAs (sRNAs) called Qrr1-4 (FIG. 9).

The main target of the Qrr sRNAs is mRNA encoding a master transcriptional regulator HapR. At LCD, the Qrr sRNAs are transcribed, and with the assistance of the RNA chaperone Hfq, these sRNAs destabilize the HapR mRNA transcript and prevent its translation. When autoinducer concentration is above the threshold level required for detection due to high cell density (HCD), autoinducers bind the cognate receptors and switches them from acting as kinases to phosphatases. Phosphate flow in the signal transduction pathway is reversed, resulting in dephosphorylation and inactivation of LuxO. Therefore, at HCD, qrr1-4s are not transcribed, HapR mRNA is stabilized, and HapR protein is produced. At high cell density, quorum sensing represses both the expression of virulence factors and the formation of biofilms. These events allow V. cholera to leave the host, re-enter the environment in large numbers and initiates a new cycle of infection.

Ultrasensitive sentinels are engineered that detect V. cholerae species specific CAI-1 by expressing codon optimized CqsS, LuxU and LuxO. FIG. 10 shows a V. cholerae sense and destroy circuit. In the absence of CAI-1, indicating low pathogen density, LuxO will be phosphorylated and promotes expression of a destabilized lambda repressor (cI-lva) [6] under P_{qrr4wt}. CI-LVA represses lambda promoter which regulates transcription of killer protein. As pathogen density increases, the concentration of CAI-1 in the medium rises, causing it to bind CqsS and trigger the phosphatase which in turn deactivates LuxO and prevents the transcription of cI-lva. CI-LVA degrades quickly, allowing killer protein expression and secretion. This construct functions as a signal amplifier that detects even minute concentrations of CAI-1 in the medium when the pathogen is still in the early stages of infection. FIG. 11 shows results obtained for a similar genetic signal amplifier circuit of a PAO-1 C₄HSL quorum sensing signal [7]. That circuit amplifies response to C₄HSL by fusing cI downstream of a rhl promoter and placing λ_{P(R)} upstream of the output yellow fluorescent protein. Since CI is a very efficient transcriptional repressor, even small increases in CI levels yield very large changes in λ_{P(R)} activity, ultimately resulting in significant changes in final output signal.

Secretion of a bacteriocin specific to V. cholerae is also engineered. Five potential bacteriocins, all synthesized in a gram-positive soil bacterium Bacillus thuringiensis, are Morricin 269, Kurstacin 287, Kenyacin 404, Entomocin 420, Tolworthcin 524[8]. These peptides have been reported to selectively kill V. cholera and are not effective against other gram-negative bacteria, including E. coli, S. typhi, S. flexneri, S. sonnei and P. aeruginosa. These bacteriocins are thermostable, resistant to α-amylase, RNAase and lysozyme, and show considerable activity at both low and high pH which is characteristic of the stomach and gut environments. They have a molecular mass between 10-25 kDa and no cysteine residues. Proteins of approximately 90 kDa have been found to be successfully secreted using FlgM.

The sensitivity of the sentinels is tested by growing them in filter sterilized supernatant of the pathogen and quantifying their response by measuring fluorescence using flow cytometry. The amount of killer protein secreted is quantified by Western Blots and Bradford Assays. Specific activity of the secreted protein is determined by characterizing the amount of purified protein required to kill a specific number of pathogen. Fine tuning analogous to that described in Example 1 is conducted to optimize sentinel response to pathogens. Once the individual parts are tested, sentinels are co-cultured with pathogen and a ratio for inhibiting the growth of V. cholera is determined.

### References for Example 2

1. Schultz, M. (2008) Clinical use of E. coli Nissle 1917 in inflammatory bowel disease, Inflammatory bowel diseases, 14(7):1012
2. Nelson, E. J. and Harris, J. B. and Morris, J. G. and Calderwood, S. B. and Camilli, A. (2009) Cholera transmission: the host, pathogen and bacteriophage dynamic, Nature, 7(10):693-702
3. Higgins, D. A. and Pomianek, M. E. and Kraml, C. M. and Taylor, R. K. and Semmelhack, M. F. and Bassler, B. L. (2007) The major Vibrio cholerae autoinducer and its role in virulence factor production, Nature, 450(7171): 883-886
4. Ng, W. L. and Bassler, B. L. (2009) Bacterial Quorum-Sensing Network Architectures, Annual Review of Genetics
5. Wingreen, N. S. and Levin, S. A. (2006) Cooperation among microorganisms, PLOS Biology 4(9): e299
6. Svenningsen, S. L. and Waters, C. M. and Bassler, B. L. (2008) A negative feedback loop involving small RNAs accelerates Vibrio cholerae's transition out of quorum-sensing mode, Genes and Development, 22(2)
7. Karig, D. and Weiss, R. (2005) Signal-amplifying genetic circuit enables in vivo observation of weak promoter activation in the Rh1 quorum sensing system, Biotechnology and bioengineering, 89(6): 709-718
8. Barboza-Corona, J. E. and Vazquez-Acosta, H. and Bideshi, D. K. and Salcedo-Hernandez, R. (2007) Bacteriocin-like inhibitor substances produced by Mexican strains of Bacillus thuringiensis, Archives of microbiology, 187(2): 117-126
9. Canton, B., Labno, A., and Endy, D. (2008) Refinement and standardization of synthetic biological parts and devices, Nature Biotech., 26: 787
10. Collins, C. H., Arnold, F. H., Leadbetter, J. (2005) Directed evolution of Vibrio fischeri LuxR for increased sensitivity to a broad spectrum of acyl-homoserine lactones, Mol. Microb., 55: 712
11. Kambam, P. K. R., et al., (2008) Directed evolution of LuxI for enhanced OHHL production, Biotech. Bioeng., 101: 263
12. Bayer, T. S., et al. (2009) Microbial conversion of biomass to methyl halides, JACS, 131:6508
13. Levskaya, A., Chevalier, A. A., Tabor, J. J., Simpson, Z. B., Layery, L. A., Levy, M., Davidson, E. A., Scouras, A., Ellington, A. D., Marcotte, E. M., & Voigt, C. A. (2005) Engineering E. coli to see light, Nature, 438: 441-442

### Example 3

### Determining the Language of Bacterial Communication Using High-Throughput DNA Synthesis and Screening

Chemical signatures that enable a programmed sentinel bacterium to recognize pathogenic bacteria and distinguish them from non-pathogens are identified. The signature of a particular pathogen ultimately consists of multiple indicators, including communication signals, metabolic byproducts, lipids, antibiotics, and toxins. Here, the focus is on an extensive screen of QS systems common to pathogenic and non-pathogenic bacteria.

An exhaustive set of ˜250 LuxIR class of quorum sensors were constructed by identifying them from sequence databases. From this set, a comprehensive set of genes for sender and receiver pairs is built using automated DNA synthesis. With a high-throughput plate-based screen, the cross-reactions between all 31,250 sender/receiver combinations is determined. This approach establishes the complete language for this class of communication signals and how it can be harnessed in a synthetic organism to identify specific bacterial species.

The canonical quorum sensor has two components: an enzyme that produces a signal (small molecule or peptide) and a sensor that responds to this signal. One of the most highly studied quorum sensors is the LuxI/LuxR pair found in Vibrio fischeri. LuxI produces the AI-1 acetylhomoserine lactone (AHL) small molecule, which diffuses freely through the membrane. It binds to LuxR, which forms a positive feedback loop by upregulating luxI transcription. LuxIR homologous are present in many diverse species. There is diversity in the R-group of the AHL and LuxR typically has high specificity for the molecule produced by its cognate LuxI. This produces a language of communication signals by which species of bacteria can recognize themselves and each other. Despite the high specificity, there is a potential for crosstalk, where LuxR has been shown to be activated by different AHLs and LuxI can produce multiple signals [9, 10, 11]. Using methods described herein, such crosstalk is identified and this information is exploited to establish more accurate communication signatures.

Many bacterial genomes have been sequenced. Currently, there are 1101 sequenced genomes in the NCBI database. By searching the genomes for LuxI homologues, ˜250 enzymes were identified that are genomically adjacent to response regulators. Over sixty species are represented within this set, including human (e.g., Pseudomonas, Burkholderia, Yersinia) and agricultural (e.g., Xanthomonas) pathogens. Along with advances in sequencing technology, the capacity for whole-gene DNA synthesis has also grown rapidly over the last few years. It is now possible to order 100 kb segments of DNA from synthesis companies (e.g., DNA 2.0, Blue Heron, GeneArt) and have the physical DNA delivered in a few weeks.

DNA synthesis is used to build the complete set of LuxI and LuxR homologs extracted from the genome sequence database. Each gene is codon optimized, if necessary, for expression in E. coli. The genes are inserted into two plasmids. The first is the sender plasmid which has luxI under the control of an IPTG-inducible promoter. The second is a receiver plasmid which contains the luxR regulator under the control of a constitutive promoter. It also has the cognate LuxR-responsive luxI promoter transcriptionally fused to the reporter gene β-galactosidase. The sender and receiver plasmids are transformed into E. coli separately to create sender and receiver cells, respectively. This yields a total of approximately 500 strains.

Communication between all sender and receiver cell combinations is screened for. A high-throughput plate-based screen is designed that rapidly identifies receiver cells responsive to sender cells. Sender and receiver cells are spotted close to each on a plate. The plates contain S-gal, which the β-gal reporter enzyme catalyzes to form a strong and stable black pigment. The screen is very sensitive and produces a graded output in response to changes in transcription [10]. Large (25×25 cm BD Falcon) plates can support 2304 colonies spaced 0.5 cm apart. Using a robot, colonies are arrayed such that sender cells communicate with neighboring receiver cells. With optimal spacing, each plate can screen seven luxI variants. Positive hits are confirmed in liquid culture experiments, where the sender and receiver pair are co-cultured and assayed for activity over time. Prior to performing the full-scale DNA synthesis, the screen is optimized using LuxR receivers from V. fischeri and P. aeruginosa and a set of eight luxI molecules known to produce AHLs that interact with these regulators [9].

This work has several significant impacts. First, synthetic biology is harnessed to exhaustively identify the language of bacterial communication for a class of signals relevant to pathogenesis. This is the first comprehensive quantification of communication channels. Characterizing these communication molecules aids in the development of sensors, including the cell-based sentinels described herein, to distinguish between bacterial species. Second, this research broadly impacts synthetic biology and the ability to program cell-cell communication by characterizing existing and discovering new channels for communication.

### References for Example 3

1. Schultz, M. (2008) Clinical use of E. coli Nissle 1917 in inflammatory bowel disease, Inflammatory bowel diseases, 14(7):1012
2. Nelson, E. J. and Harris, J. B. and Morris, J. G. and Calderwood, S. B. and Camilli, A. (2009) Cholera transmission: the host, pathogen and bacteriophage dynamic, Nature, 7(10):693-702
3. Higgins, D. A. and Pomianek, M. E. and Kraml, C. M. and Taylor, R. K. and Semmelhack, M. F. and Bassler, B. L. (2007) The major Vibrio cholerae autoinducer and its role in virulence factor production, Nature, 450(7171): 883-886
4. Ng, W. L. and Bassler, B. L. (2009) Bacterial Quorum-Sensing Network Architectures, Annual Review of Genetics
5. Wingreen, N. S, and Levin, S. A. (2006) Cooperation among microorganisms, PLOS Biology 4(9): e299
6. Svenningsen, S. L. and Waters, C. M. and Bassler, B. L. (2008) A negative feedback loop involving small RNAs accelerates Vibrio cholerae's transition out of quorum-sensing mode, Genes and Development, 22(2)
7. Karig, D. and Weiss, R. (2005) Signal-amplifying genetic circuit enables in vivo observation of weak promoter activation in the Rh1 quorum sensing system, Biotechnology and bioengineering, 89(6): 709-718
8. Barboza-Corona, J. E. and Vazquez-Acosta, H. and Bideshi, D. K. and Salcedo-Hernandez, R. (2007) Bacteriocin-like inhibitor substances produced by Mexican strains of Bacillus thuringiensis, Archives of microbiology, 187(2): 117-126
9. Canton, B., Labno, A., and Endy, D. (2008) Refinement and standardization of synthetic biological parts and devices, Nature Biotech., 26: 787
10. Collins, C. H., Arnold, F. H., Leadbetter, J. (2005) Directed evolution of Vibrio fischeri LuxR for increased sensitivity to a broad spectrum of acyl-homoserine lactones, Mol. Microb., 55: 712
11. Kambam, P. K. R., et al., (2008) Directed evolution of LuxI for enhanced OHHL production, Biotech. Bioeng., 101: 263
12. Bayer, T. S., et al. (2009) Microbial conversion of biomass to methyl halides, JACS, 131:6508
13. Levskaya, A., Chevalier, A. A., Tabor, J. J., Simpson, Z. B., Layery, L. A., Levy, M., Davidson, E. A., Scouras, A., Ellington, A. D., Marcotte, E. M., & Voigt, C. A. (2005) Engineering E. coli to see light, Nature, 438: 441-442

### Example 4

### Shigella Sense-and-Destroy

Shigella is a Gram-negative bacterium that is nonmotile and facultatively anaerobic, shaped as non-spore-forming rods. It is the principal agent of bacillary dysentery also called shigellosis. Three Shigella groups out of four are the major disease-causing species: S. flexneri is the most frequently isolated species worldwide and accounts for 60% of cases in the developing world; S. sonnei causes 77% of cases in the developed world, compared to only 15% of cases in the developing world; and S. dysenteriae is usually the cause of epidemics of dysentery. The serotype 1 of S. dysenteriae (Sd1) is of particular concern due to its expression of the Shiga toxin (Stx). It is the cause of epidemic dysentery and can cause vicious outbreaks in confined populations. Stx inhibits protein synthesis in eukaryotic cells via inactivation of ribosomal RNA, leading to cell death. The toxin is cytotoxic, neurotoxic and enterotoxic. It targets glomerular epithelial cells, central nervous system and microvascular endothelial cells causing haemolytic-uremic syndrome (HUS) and seizures. Sd1 also causes a rapid increase in the cell membrane permeability of infected macrophages and destroys their mitochondrial function. A major obstacle to the control of Sd1 is its resistance to antimicrobial drugs.

A programmable sense-and-destroy system is engineered (FIG. 15) wherein E. coli sentinels detect Shigella using its QS signal AI-3 along with its lambdoid phage and then specifically kill the pathogen without releasing the toxin out of the dead cells and thus reducing Shigella infection.

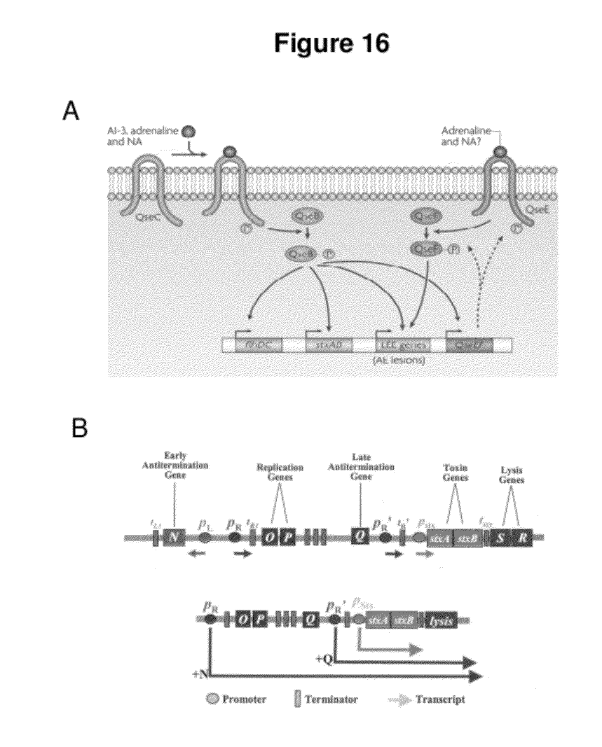

It has been calculated that human gastrointestinal tract houses 10¹⁴ bacteria. The proximal small intestine has a relatively sparse Gram-positive flora, consisting mainly of lactobacilli and Enterococcus faecalis. This region has about 10⁵-10⁷ bacteria per ml of fluid. The distal part of the small intestine contains greater numbers of bacteria (10⁸/ml) and additional species, including coliforms (E. coli and relatives) and Bacteroides, in addition to lactobacilli and enterococci. This is the neighborhood of Campylobacter, nontyphoid Salmonella, Shiga-Toxin producing E. coli and Shigella (the most common cause of bloody diarrhea). A non-pathogenic commensal strain of E. coli Nissle is engineered which lives in the same environment to detect Shigella first by recognizing one of its QS signal. Sentinels express high amount of transmembrane histidine kinase (HK) QseC to detect autoinducer AI-3. AI-3 is a known bacterial signal that binds the bacterial membrane receptor QseC and results in its auto-phosphorylation (FIG. 16).

Shigella requires very low quantity of inoculum (10⁶-10⁷ CFU) for clinical manifestations of Acute Gastrointestinal Infections [25]. Without wishing to be bound by any theory, this could be because the QS response regulator QesC is very sensitive and even a small amount of signal activates full autophosphorylation for a quick response [19]. 100 nM of AI-3 has been shown to invoke a response from QseC in vivo [19]. QseC then phosphorylates its response regulator QseB and results in expression of the Shigella and Salmonella virulence genes [18, 12]. AI-3 defective cells are unable to colonize and cause pathogenicity [8]. AI-3 is produced by several species of bacteria in the normal human GI microbial flora but many of them exist either in respiratory tract, urinary tract or distal colon. Enteric pathogens, including Shigella and Salmonella, occupy the distal part of small intestine and early colon where there is relatively less gut flora to crosstalk with our engineered sensing. The sentinels themselves carry a mutation in luxS gene making them defective in producing AI-3 hence preventing crosstalk (luxS mutations are not lethal for the cell).

In principle, AI-3 sensing is not needed for system operation but can be advantageous for the metabolic fitness of sentinels, especially for future multi-input sense and destroy cells. AI-3 sensing also prepares the sentinels for the possibility of an attack. Most antibiotic therapies are ineffective because by the time symptoms of a particular disease appear it is already too late (e.g. for V. cholera). Instead, sentinels described herein launch an early response and destroy the pathogen even before the symptoms are present. Since AI-3 is not sufficient proof of Shigella existence, once sentinels detect AI-3 they will employ a two-pronged approach to more specifically detect and destroy the pathogen with minimum damage to enterocytes and neighboring gut flora.

First, sentinel killer cells express molecular mimics of Shiga toxin (Stx1/Stx2) receptors (Gb3) on the surface to sequester the toxin which ‘may’ be present, assuming the pathogen is there, into the lumen of intestine. The Stx family, a group of structurally and functionally related exotoxins, includes Stx from Shigella dysenteriae serotype 1 and the Shiga toxins that are produced by enterohaemorrhagic Escherichia coli (EHEC) strains. These toxins can be Stx1 variants (Stx1 and Stx1c), Stx2 variants (Stx2, Stx2c, Stx2d, Stx2e, and Stx2f) or variants of both in a range of combinations. Gb3 (Saccharide structure: Gal (α1, 4) Gal (β1, 4) Glcβ1- -) is the primary natural glycoconjugate receptor for Stx1/Stx2 on enterocytes, the main colonizing factor of Shigella Dysenteriae. In [10, 11] it is demonstrated that these receptors neutralize more than 98% of the cytotoxicity of each of the Stx types associated with human disease.

Chimeric LPS is incorporated into the outer membrane of the sentinels. With a mutation in the waaO gene, LPS core is truncated and terminates in Glc. Insertion of two Neisseria galactosyl-transferase genes (lgtC and lgtE) directs the addition of two Gal residues to the Glc acceptor, generating a chimeric LPS terminating in Gal(α1, 4)Gal(β1, 4)Glc, which is the Stx receptor. This in turn prevents Stx from binding similar glycolipid receptors on the surface of enterocytes and their characteristic attaching and effacing (A/E) histology. Second, sentinels express Shigella lambdoid phage specific receptor, YaeT, [6, 7] to absorb phage containing the virulence genes and Stx genes. The toxins in S. dysenteriae are encoded by diverse temperate lambdoid bacteriophages. These phages are highly mobile genetic elements that play an important part in horizontal gene transfer. Infection of E. coli by Shiga toxin-encoding bacteriophages (Stx phages) was the pivotal event in the evolution of the deadly Shiga toxin-encoding E. coli (STEC), of which serotype O157:H7 is the most notorious. The number of different bacterial species and strains reported to produce Shiga toxin is now more than 500 after the first reported STEC infection outbreak in 1982.

In the sense-and-destroy system described herein, incoming phage, along with AI-3, provides the sentinels sufficient proof of Shigella existence. After infection (FIG. 16), phage repressor silences transcription of most of its genes [17, 3, 2]. Removal of repression leads to a cascade of regulatory events beginning with expression of N transcription antitermination protein. Terminator read through mediated by the N protein results in expression of delayed early genes that encode products involved in replication, prophage excision and expression of late genes which include Stx genes. Thus Stx expression by lambdoid prophages is a consequence of phage cycle. Sentinels sense the lytic phase of the incoming phage by having the same phage promoter P_{L}, activated by N protein, control phage and pathogen killing.

Based on system performance, a positive feedback regulator can be added on P_{L} after phage detection to maintain FlgM-Bacteriocin synthesis for a while until the pathogen is effectively destroyed.

Once phage enters the lytic phase, sentinels immediately express Clustered Regularly Interspaced Short Palindromic Repeats (CRISPRs) sequences [4, 9, 13, 14, 15, and 21] specific to incoming phage DNA and Shigella specific bacteriocin on a high copy number plasmid. Engineered CRISPRs have been shown to confer phage resistance [13, 14, and 15]. CRISPRs are small repeated sequences separated by short spacer sequences that match bacteriophage and specify the targets of interference, a mechanism similar but not homologous to RNAi in eukaryotes (FIG. 17). The repeat-spacer array is transcribed into a long RNA, and the repeats assume a secondary structure. Cas (CRISPR-associated) proteins naturally present in the sentinel/killer cells recognize the sequence or structure of the repeats and process the RNA to produce small RNAs (sRNAs), each of which contains a spacer and two half repeats. The sRNAs, complexed with additional Cas proteins, base-pair with phage nucleic acids, leading to their degradation. CRISPR is engineered to target genes of the phage, lytic gene lys, Shiga toxin gene Stx, and replication and proliferation genes o and p. Having P_{L} on a high copy number plasmid further helps titrate away N and prevents expression of phage genes before CRISPR.

Besides destroying phage and reducing its spread, it is imperative to kill the pathogen safely. Conventional anti-microbial therapies are counterproductive since killing the bacteria may accelerate toxin release [27]. Hence, herein, the bacteria are killed without lysis in order to prevent toxin release and septic shock from the LPS outer membrane. This issue is addressed by coupling secretion of engineered Shigella specific colicin (Colicin U [22, 23]) with CRISPR expression. Colicins are bacteriocins produced by certain bacterial strains of the family Enterobacteriaceae, and their toxic effects are limited to sensitive strains within the species of the producer strain. Group A, to which Colicin U belongs [22, 23], have modular three-domain architecture. Their production is strictly regulated and coordinated with production of an Immunity Protein which provides immunity to the producing cell by binding and neutralizing colicin Killing Domain.

Once the colicins are released into the extracellular space, the Receptor Domain of the bacteriocin binds a specific receptor on the outer membrane of the target cell. Then the Translocase Domain forms a complex with the tol receptors on the surface of the cell and facilitates release of the Immunity Protein bound to the Killing/Nuclease Domain. The Killing Domain then enters the target cell and degrades the DNA/RNA without disrupting the outer membrane and hence this Shigella antimicrobial approach reduces the possibility of septic shock. The Receptor and Translocase Domain of colicin U are fused to the nuclease and immunity domain of colicin E3 produced by E. coli. This allows the new hybrid colicin, CoShi, to recognize and specifically kill Shigella strains while leaving the producing strain unharmed.

The response sensitivity of the sentinels is tested by growing them in filter sterilized supernatant of the pathogen (>10² per ml for clinical relevance) and quantifying their response (in some embodiments, with a response goal of approximately 100 nM AI-3) by measuring fluorescence using flow cytometry. Immunofluorescent staining and epi-fluorescent microscopy are used to assay sequestering of Stx by the sentinels [10, 11] and expression of YaeT on the surface of sentinels. Phage immunity and sensitivity are measured by cfu counting and efficiency of plaquing [6]. The amount of killer protein secreted is quantified by Western Blots and Bradford Assays. Specific activity of the secreted protein is determined by characterizing the amount of purified protein required to kill a specific number of pathogen. Fine tuning is used to optimize sentinel response to pathogens.

System architecture described herein is highly modular and every single module is responsible for addressing a different aspect of the pathogen. Hence the system is still useful even before all modules have been made functional and fully optimized. Once the individual parts are validated, sentinels are co-cultured with pathogen and a ratio for inhibiting the growth of Shigella in vitro with Vero/Caco-2 cell lines is determined. Microfluidic GI tract models are used to accurately predict spatiotemporal parameters. In vivo Human Intestinal Xenograft Infection [24] models are used to test the efficacy of the system.

### References for Example 4

1. Son, M. S. and Matthews Jr, W. J. and Kang, Y. and Nguyen, D. T. and Hoang, T. T. 2007, Infection and immunity, p. 5313.
2. Wagner, P. L., et al. 2001, Journal of Bacteriology, Vol. 183, p. 2081.
3. Wagner, P. L. and Waldor, M. K. 2002, Infection and immunity, Vol. 70, p. 3985.
4. Sorek, R., Kunin, V. and Hugenholtz, P. 2008, Nature Reviews Microbiology, Vol. 6, pp. 181-186.
5. Smith, R. S., et al. 2002, Journal of bacteriology, Vol. 184, p. 1132.
6. Smith, D. L., et al. 2007, Journal of bacteriology, Vol. 189, p. 7223.
7. Schmidt, H. 2001, Research in microbiology, Vol. 152, pp. 687-695.
8. Rasko, D. A., et al. 2008, Science, Vol. 321, p. 1078.
9. Pul, 2010, Molecular Microbiology, Vol. 9999.
10. Paton, A. W., Morona, R. and Paton, 2006, Nature Reviews Microbiology, Vol. 4, pp. 193-200.
11. Paton, A. W., Morona, R. and Paton, J. C. 2000, Nature Medicine, Vol. 6, pp. 265-270.
12. Moreira, C. G., Weinshenker, D. and Sperandio, V. 2010, Infection and Immunity, Vol. 78, p. 914.
13. Marraffini, L. A. and Sontheimer, E. J. 2010, Nature, Vol. 463, pp. 568-571.
14. Marraffini, L. A. and Sontheimer, E. J. 2010, Nature Reviews Genetics, Vol. 11, pp. 181-190.
15. Labrie, S. J., Samson, J. E. and Moineau, S. 2010, Nature Reviews Microbiology.
16. Karig, D. and Weiss, R. 2005, Biotechnology and bioengineering, Vol. 89, pp. 709-718.
17. James, C. E., et al. 2001, Applied and environmental microbiology, Vol. 67, p. 4335.
18. Hughes, D. T. and Sperandio, V. 2008, Nature Reviews Microbiology, Vol. 6, pp. 111-120.
19. Clarke, M. B., et al. 2006, Proceedings of the National Academy of Sciences, Vol. 103, p. 10420.
20. Chun, C. K., et al. 2004, Proceedings of the National Academy of Sciences, Vol. 101, p. 3587.
21. Barrangou, R., et al. 2007, Science, Vol. 315, p. 1709.
22. Smajs, D., Pilsl, H. and Braun, 1997, Journal of bacteriology, Vol. 179, p. 4919.
23. Cascales, E., et al. 2007, Microbiology and Molecular Biology Reviews, Vol. 71, p. 158.
24. Sperandio, B., et al. 2008, Journal of Experimental Medicine, Vol. 205, p. 1121.
25. De, L L Van, et al. 1995, Infection and immunity, Vol. 63, p. 1947.
26. Huh, D., et al. 2007, Proceedings of the National Academy of Sciences, Vol. 104, p. 18886.
27. Johannes, L. and Romer, W. 2009, Nature Reviews Microbiology, Vol. 8, pp. 105-116.

### EQUIVALENTS

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Those skilled in the art will recognize, or be able to ascertain using no more than routine experimentation, many equivalents to the specific embodiments of the invention described herein. Such equivalents are intended to be encompassed by the following claims. All references, including patent documents, disclosed herein are incorporated by reference in their entirety, particularly for the disclosure referenced herein.

What is claimed is:

1. A cell that recombinantly expresses (1) a detection circuit, (2) optionally a signal amplifying circuit, and (3) a secretion circuit that secretes a factor that specifically recognizes and destroys a specific pathogen.

2. The cell of claim 1, wherein the cell is a bacterial cell, optionally wherein the bacterial cell is an Escherichia coli (E. coli) cell, such as an E. coli Nissle 1917 (EcN) cell.

3.-5. (canceled)

6. The cell of claim 1, wherein the cell is an algal cell, a fungal cell (including a yeast cell), an insect cell or an animal cell, optionally wherein the cell is a mammalian cell and optionally wherein the cell is a T cell or a B cell.

7.-10. (canceled)

11. The cell of claim 1, wherein the cell detects one or more molecules produced by one or more specific pathogens, optionally wherein one or more of the specific pathogens is a bacterial pathogen and/or wherein one or more of the molecules produced by one or more of the specific pathogens is a quorum sensing molecule.

12.-13. (canceled)

14. The cell of claim 11, wherein the bacterial pathogen is a Pseudomonas bacterial pathogen, optionally a Pseudomonas aeruginosa bacterial pathogen.

15. (canceled)

16. The cell of claim 14, wherein the cell detects 3-oxo-C12-homoserine lactone (3OC12HSL) that is secreted by the Pseudomonas aeruginosa bacterial pathogen, and optionally wherein the cell comprises a transcriptional regulator that regulates expression of one or more genes in response to 3OC12HSL.

17.-23. (canceled)

24. The cell of claim 11, wherein the bacterial pathogen is a Vibrio bacterial pathogen, optionally a Vibrio cholerae bacterial pathogen.

25. (canceled)

26. The cell of claim 24, wherein the cell detects CAI-1 that is secreted by the Vibrio cholerae bacterial pathogen, and optionally wherein the cell comprises a signal amplifying circuit that regulates production of a factor that is secreted by the cell to specifically destroy the Vibrio bacterial pathogen.

27. (canceled)

28. The cell of claim 26, wherein the cell expresses CqsS, LuxU and/or LuxO, optionally wherein CqsS, LuxU and/or LuxO are codon-optimized.

29.-30. (canceled)

31. The cell of claim 1, wherein the pathogen is a viral pathogen or a fungal pathogen.

32.-36. (canceled)

37. The cell of claim 1, wherein the factor that is secreted by the cell is a bacteriocin, optionally wherein the bacteriocin is a lysin, and optionally wherein a flagellar gene, or a portion thereof, is expressed as a fusion with the lysin in the cell.

38.-44. (canceled)

45. The cell of claim 37, wherein the lysin is a colicin or a pyocin, optionally wherein the colicin or pyocin comprises a chimeric recognition domain that is modified such that the colicin or pyocin specifically recognizes and destroys a specific pathogen.

46.-51. (canceled)

52. The cell of claim 37, wherein the cell secretes a bacteriocin that is specific for V. cholera, optionally wherein the bacteriocin is selected from the group consisting of Morricin 269, Kurstacin 287, Kenyacin 404, Entomocin 420 and Tolworthcin 524.

53.-55. (canceled)

56. The cell of claim 11, wherein the bacterial pathogen is a Shigella bacterial pathogen or a Salmonella bacterial pathogen, optionally wherein the bacterial pathogen is a Shigella dysenteriae bacterial pathogen, and optionally wherein the cell detects AI-3 and its lambdoid phage.

57.-58. (canceled)

59. The cell of claim 56, wherein the cell expresses QseC and QseB, one or more molecular mimics of a Shiga toxin receptor, a chimeric LPS in the outer membrane of the cell, the Shigella lambdoid phage specific receptor (YaeT), one or more Clustered Regularly Interspaced Short Palindromic Repeats (CRISPRs) sequences specific to incoming phage, and/or a Shigella specific bacteriocin.

60.-68. (canceled)

69. The cell of claim 1, wherein the cell is a component of a probiotic, a pharmaceutical preparation, a vaccine, a food or nutraceutical, a biospray and/or a water supply system.

70.-74. (canceled)

75. A method for destroying a specific pathogen, the method comprising,
providing a cell that recombinantly expresses (1) a detection circuit, (2) optionally a signal amplifying circuit, and (3) a secretion circuit that secretes a factor that specifically recognizes and destroys the specific pathogen.

76. The method of claim 75, wherein the cell is a bacterial cell, optionally wherein the bacterial cell is an Escherichia coli (E. coli) cell such as an E. coli Nissle 1917 (EcN) cell.

77.-79. (canceled)

80. The method of claim 75, wherein the cell is an algal cell, a fungal cell (including a yeast cell), an insect cell or an animal cell, optionally wherein the cell is a mammalian cell and optionally wherein the cell is a T cell or a B cell.

81.-84. (canceled)

85. The method of claim 75, wherein the cell detects one or more molecules produced by one or more specific pathogens, optionally wherein one or more of the specific pathogens is a bacterial pathogen and/or wherein one or more of the molecules produced by one or more of the specific pathogens is a quorum sensing molecule.

86.-87. (canceled)

88. The method of claim 85, wherein the bacterial pathogen is a Pseudomonas bacterial pathogen, a Vibrio cholerae bacterial pathogen, a Shigella bacterial pathogen or a Salmonella bacterial pathogen, optionally wherein the bacterial pathogen is a Shigella dysenteriae bacterial pathogen.

89.-142. (canceled)

143. The method of claim 75, wherein the cell is a component of a probiotic, a pharmaceutical preparation, a vaccine, a food or nutraceutical, a biospray and/or a water supply system.

144.-148. (canceled)

149. The method of claim 75, wherein the method is a method of treating or preventing a disease, optionally wherein the disease is an infectious disease, and optionally wherein the cell is provided in a therapeutically effective amount to a subject in need thereof.

150.-151. (canceled)

152. The method of claim 75, wherein the pathogen is within the gastrointestinal tract and/or within the lung of a subject.

153. (canceled)

154. The method of claim 75, wherein the method is a method of sterilization, optionally wherein the sterilization comprises sterilization of medical equipment.

155. (canceled)

156. A method for protecting against a specific pathogen, the method comprising,
administering to a subject in need thereof an effective amount of a composition comprising a cell that recombinantly expresses (1) a detection circuit, (2) optionally a signal amplifying circuit, and (3) a secretion circuit that secretes a factor that specifically recognizes and destroys the specific pathogen.

157.-223. (canceled)

224. The method of claim 156, wherein the cell is a component of a probiotic, a pharmaceutical preparation, a vaccine, a food or nutraceutical, a biospray and/or a water supply system.

225.-229. (canceled)

230. The method of claim 156, wherein the method is a method of treating or preventing a disease, optionally wherein the disease is an infectious disease.

231.-236. (canceled)
